(19) United States
(12) Patent Application Publication
(10) Pub. No.: US 2015/0344836 A1
(43) Pub. Date: Dec. 3, 2015

(54) Agrobacterium Strains for Plant Transformation and Related Materials and Methods

(71) Applicant: Ohio State Innovation Foundation, Columbus, OH (US)

(72) Inventors: John James Finer, Wooster, OH (US); Kyle Arthur Benzle, Hilliard, OH (US); Kim R. Finer, Wooster, OH (US); Christopher G. Taylor, Wooster, OH (US)

(73) Assignee: OHIO STATE INNOVATION FOUNDATION, Columbus, OH (US)

(21) Appl. No.: 14/725,901

(22) Filed: May 29, 2015

Related U.S. Application Data
(60) Provisional application No. 62/005,204, filed on May 30, 2014.

(51) Int. Cl. C12N 1/20; C12N 15/82

(52) U.S. Cl. CPC C12N 1/20; C12N 15/8205

(57) ABSTRACT

Described herein are materials, methods, and kits useful for Agrobacterium-mediated transformation of plant cells and plants. In particular, the present disclosure provides a novel strain of Agrobacterium and its disarmed variant. The present disclosure further provides methods and kits for transforming plant cells and plants utilizing the novel strains of Agrobacterium.

### CROSS-REFERENCE TO RELATED APPLICATIONS

This application claims priority to U.S. Provisional Application No. 62/005,204, filed on May 30, 2014, the entire disclosure of which is expressly incorporated herein by reference for all purposes.

### STATEMENT REGARDING FEDERALLY SPONSORED RESEARCH

This invention was not made with government support.

### SEQUENCE LISTING

The instant application contains a Sequence Listing which has been submitted via EFS-web in ASCII format and is hereby incorporated by reference in its entirety. The ASCII copy, created on May 29, 2015, is named 1-56161-OSIF-2014-232_SL.txt and is 2,265 bytes in size.

### BACKGROUND OF THE INVENTION

Members of the genus Agrobacterium cause the neoplastic diseases crown gall (A. tumefaciens and A. vitis), hairy root (A. rhizogenes), and cane gall (A. rubi) on numerous plant species. Researchers have identified key bacterial genes involved in virulence, and genomic technologies have revealed numerous additional bacterial genes that more subtly influence transformation. The results of these genomic analyses allowed scientists to develop a more integrated view of how Agrobacterium interacts with host plants. Similarly, numerous plant genes important for Agrobacterium-mediated genetic transformation have been identified. Knowledge of these genes and their roles in transformation has revealed how Agrobacterium manipulates its hosts to increase the probability of a successful transformation outcome.

Virulent strains of Agrobacterium contain tumor-inducing (Ti) or root-inducing (Ri) plasmids. During infection, proteins encoded by virulence (vir) genes process the T-DNA region of these plasmids. The resulting single-stranded DNA (T-strand) linked to VirD2 protein exits the bacterium via a type IV protein secretion system and enters the plant cell. Within the plant, T-strands likely form complexes with other secreted virulence effector proteins, including VirE2, VirE3, VirD5, and VirF, and supercomplexes with plant proteins as they traverse the cytoplasm and target the nucleus. Once inside the nucleus, T-strands integrate into the plant genome and express T-DNA-encoded transgenes. Two classes of T-DNA genes mediate the pathology of Agrobacterium infection. The first group—the oncogenes—either effect phytohormone production, sensitize the plant to endogenous hormone levels, or may be involved in chromatin remodeling. Expression of these genes results in formation of either galls or hairy roots. A second set of genes directs the synthesis of various opines that can serve as energy sources for the inciting bacterial strain and can perhaps affect virulence.

Agrobacterium is well-known as an agent of horizontal gene transfer that plays an essential role in basic scientific research and in agricultural biotechnology. In the 1980s, scientists learned to disarm (by deleting the oncogenes and, usually, the opine synthase genes) virulent Agrobacterium strains such that tissues transformed by the bacteria could regenerate into normal plants, free of the oncogenes that caused either gall growth or hairy root formation. Inserting genes of interest (transgenes) in the place of oncogenes and opine synthase genes resulted in plants expressing these genes of interest and, thus, novel phenotypes. Although initially conducted in cis (i.e. transgenes were placed within T-DNA of native Ti-plasmids), the development of binary systems, in which T-DNA and virulence helper plasmids were separated into two different vectors, greatly increased the utility of Agrobacterium as a vehicle for gene transfer.

The binary vector systems offer a great degree of flexibility, since they do not require a specifically engineered Ti plasmid with a homologous recombination site. A disarmed Agrobacterium strain, wherein the T-DNA region is modified or removed completely, can be used to transfer genes for any binary vector. Due to their versatility, binary vectors are the preferred intermediate vectors for cloning genes destined for Agrobacterium-mediated transformation in plants. However, it is preferable that strains of Agrobacterium to be used with binary vectors have its own disarmed Ti plasmid, especially if the target plant species in inefficiently transformed by Agrobacterium. Otherwise, the gene(s) of interest from the binary vector will be co-transformed along with the tumor-inducing genes from the native T-DNA of the bacteria, possibly reducing transformation efficiency of the target gene(s) and also producing tumorigenic disease symptoms in many of the target cells, thereby preventing differentiation of these cells into normal plants.

Agrobacterium has a diverse dicot host range, and additionally some monocot families. There are several different strains of Agrobacterium. A major disadvantage of using Agrobacterium for plant transformation is the organism's host specificity, resulting in low levels of transformation in certain plant species. Soybean (Glycine max) has proven to be very difficult to transform with Agrobacterium. This is at least in part because it is refractory to infection by known strains of A. tumefaciens. Studies with a number of soybean cultivars and different Agrobacterium strains have suggested that soybean susceptibility to Agrobacterium is limited, and may be both cultivar- and bacterial strain dependent. One strain, A281, is a supervirulent, broad host-range, L,L-succinamopine-type A. tumefaciens with a nopaline-type C58 chromosomal background, containing the L,L-succinamopine-type Ti plasmid, pTiBo542. Disarming this strain has produced EHA101 and EHA105, strains now widely used in conjunction with soybean transformation.

Unfortunately, transformation of soybean using even these strains remains inefficient. There is still a significant need for improved or novel strains of Agrobacterium capable of effectively and efficiently transforming soybean. Therefore, it was an objective of the present invention to provide novel strains of Agrobacterium having improved transformation efficiency in a wide variety of plants, including soybean. Further objectives of the present invention were to provide disarmed variants of novel strains, and methods for the use of the novel strains in Agrobacterium-mediated transformation.

### SUMMARY OF THE INVENTION

Described herein are materials, methods, and kits useful for Agrobacterium-mediated transformation of plant cells and plants. In particular, the present disclosure provides a novel strain of Agrobacterium and its disarmed variant. The present disclosure further provides methods and kits for transforming plant utilizing the novel strains of Agrobacterium.

In a particular embodiment described herein is an isolated Agrobacterium strain JTND, a deposit of which is maintained by Dr. John J. Finer, Department of Horticulture and Crop Science, The Ohio State University, 1680 Madison Ave., Wooster, Ohio 44691.

In another particular embodiment provided herein, the isolated Agrobacterium strain JTND is substantially biologically pure. In another particular embodiment provided herein, the isolated Agrobacterium strain JTND is disarmed.

In another particular embodiment described herein is a disarmed Agrobacterium strain SBHT, a deposit of which is maintained by Dr. John J. Finer, Department of Horticulture and Crop Science, The Ohio State University, 1680 Madison Ave., Wooster, Ohio 44691.

In another particular embodiment provided herein, the isolated Agrobacterium strain SBHT is substantially biologically pure.

In a particular embodiment described herein is a method of producing a transgenic plant cell, transgenic plant tissue, or transgenic plant, comprising the steps: a) providing an Agrobacterium strain selected from the group consisting of: JTND; and SBHT, wherein the Agrobacterium strain comprises a transgenic T-DNA region; and b) contacting the Agrobacterium with a plant cell, plant tissue, or plant under conditions that permit the Agrobacterium to transform the plant cell, plant tissue, or plant, thereby producing a transgenic plant cell, transgenic plant tissue, or transgenic plant. In certain embodiments, the transgenic T-DNA comprises at least one plant-expressible gene of interest. In other embodiments, the at least one plant-expressible gene of interest comprises one or more genes associated with at least one agronomically valuable trait selected from the group consisting of: increased yield; drought tolerance; cold tolerance; heat tolerance; salt tolerance; increased nutrient content; reduced development time; increased vigor; herbicide resistance; and pest resistance.

In another particular embodiment provided herein, the method further comprises isolating or selecting a plant cell, plant tissue, or plant comprising the transgenic T-DNA.

In another particular embodiment provided herein, the transgenic T-DNA is stably integrated into the genome of the plant cell, plant tissue, or plant.

In another particular embodiment provided herein, the method further comprises regenerating a plant from the transgenic plant cell or plant tissue comprising the transgenic T-DNA.

In another particular embodiment provided herein, the transgenic T-DNA region comprises at least one plant-expressible selectable marker gene.

In another particular embodiment provided herein, the step of contacting the Agrobacterium with the plant cell, plant tissue, or plant is accomplished by at least one method selected from the group consisting of: incubating the plant cell, plant tissue, or plant with Agrobacterium; co-cultivation of the at least one plant host cell and the Agrobacterium; floral dip method; vacuum infiltration method; cotyledonary-node method; and sonication-assisted Agrobacterium-mediated transformation, or a combination thereof.

In a particular embodiment provided herein is a transgenic plant comprising a plurality of transgenic plant cells produced by a method described herein.

In another particular embodiment provided herein, the plant tissue is selected from the group consisting of: immature plant embryo; mature plant embryo; seed; seedling; root; cotyledon; stem; node; internode; bud; leaf; shoot apical meristem; and cultured plant material.

In another particular embodiment provided herein, the plant cell is from a member of the group consisting of: pollen; ovule; immature plant embryo; mature plant embryo; seed; seedling; root; cotyledon; stem; node; internode; bud; leaf; shoot apical meristem; and cultured plant material.

In another particular embodiment provided herein, the plant cell or plant tissue is from a plant selected from the group consisting of: monocotyledonous plants; dicotyledonous plants; and gymnosperm plants.

In another particular embodiment provided herein, the plant is selected from the group consisting of: monocotyledonous plants; dicotyledonous plants; and gymnosperm plants.

In another particular embodiment provided herein, the plant cell or plant tissue is from a plant of a genus selected from the group consisting of: Glycine; Medicago; Pisum; Beta, Helianthus; Arabidopsis; Dioscorea; Ipomea; Manihot; Plantago; Zea; Oryza; Sorghum; Triticum; Hordeum; Saccharum; Brassica; Solanum; Nicotiana; Gossypium; Vitis; Populus; Picea; and Pinus.

In another particular embodiment provided herein, the plant is of a genus selected from the group consisting of: Glycine; Medicago; Pisum; Beta, Helianthus; Arabidopsis; Dioscorea; Ipomea; Manihot; Plantago; Zea; Oryza; Sorghum; Triticum; Hordeum; Saccharum; Brassica; Solanum; Nicotiana; Gossypium; Vitis; Populus; Picea; and Pinus.

In another particular embodiment provided herein, the plant cell or plant tissue is from a plant of the genus Glycine.

In another particular embodiment provided herein, the plant cell or plant tissue is from a soybean plant.

In another particular embodiment provided herein, the plant is of the genus Glycine.

In another particular embodiment provided herein, the plant is a soybean plant.

In another particular embodiment provided herein, a method described herein further comprises selecting a transgenic plant or progeny thereof having an agronomically valuable trait conferred by the transgenic T-DNA region.

In another particular embodiment provided herein, seeds are obtained from the plant comprising the transgenic T-DNA region.

In another particular embodiment provided herein, the seeds comprise the transgenic T-DNA region.

In another particular embodiment provided herein, a method described herein further comprises a) selfing the transgenic plant or crossing the transgenic plant with a second plant; and b) selecting resulting progeny comprising the transgenic T-DNA region.

In a particular embodiment described herein is a kit for transforming a plant cell, plant tissue, or plant comprising: at least one sample comprising an Agrobacterium strain selected from the group of Agrobacterium strains consisting of: JTND; and SBHT, wherein the Agrobacterium strain comprises a Ti-plasmid.

In a particular embodiment described herein is a kit for transforming a plant cell, plant tissue, or plant comprising: a) at least one sample comprising an Agrobacterium strain selected from the group of Agrobacterium strains consisting of: JTND; and SBHT, wherein the Agrobacterium strain comprises a helper plasmid, wherein the helper plasmid comprises a Ti plasmid comprising a vir region of the Agrobacterium, and wherein the helper plasmid lacks a T-DNA region; and b) at least one sample comprising a binary plasmid, wherein the binary plasmid comprises a T-DNA region.

In another particular embodiment described herein, a kit described herein further comprises at least one sample of growth media.

In another particular embodiment described herein, a kit described herein further comprises at least one sample of at least one antibiotic, wherein the at least one antibiotic is capable of eliminating the Agrobacterium strain following transformation of a plant cell.

In another particular embodiment described herein, a kit described herein further comprises at least one sample of at least one reagent capable of selecting for transgenic plant cells following transformation of a plant cell.

In another particular embodiment described herein, a kit described herein further comprises instructions for the use of the kit.

The present invention also provides methods to make host-enhanced transformation tools, comprising: obtaining a soil sample from a field where a host plant has grown or is growing; isolating an Agrobacterium strain from the soil sample; and disarming the Agrobacterium strain so as to make a host-enhanced transformation tool.

Also provided are such methods, which further comprise introducing foreign nucleic acid into the Agrobacterium strain.

Also provided are products made according to the methods herein.

Also provided are methods to transfer foreign nucleic acid into a host plant, comprising introducing a transformation tool herein to a host plant, and inducing the Agrobacterium strain to transfer the foreign nucleic acid to the host plant.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIGS. 1A-1M: Transformation in sunflower hypocotyl tissues using known and novel strains. A degree of tissue specific transformation is seen in C58 derived strains, in contrast to the even distribution of transformed cells in most novel strains, including JTND. a) EHA105, b) C58, c) J2, d) K599, e) BGOH, f) CTOHc, g) CTOHr, h) CTOHb, i) DSOH, j) EROH, k) JTND, l) KFOH, m) SDOH.

FIG. 2: Tissue-specific transformation of 5 day old sunflower seedling hypocotyl explants. Three known strains containing the C58 chromosomal background, C58, EHA105 and J2, all showed preferential transformation of vascular tissues. Novel isolates did not show a significant difference in type of tissue transformed. Data are mean±standard error (SE) from at least three independent experiments.

FIG. 3A: GFP foci counts in 5 day old sunflower seedling hypocotyl explants. Data are mean±standard error (SE) from at least three independent experiments. Target material was inoculated, co-cultured for 48 h and then transferred to an antibiotic-containing medium for control of Agrobacterium growth. After an additional 72 h, transformation efficiency was quantified by counting expressing foci per explant. Sunflower hypocotyl explants showed that strain EHA105 had the highest transformation.

FIG. 3B: Soybean hypocotyl transformation of 5 day old soybean seedling explants. Data are mean±standard error (SE) from at least three independent experiments.

FIG. 3C: Soybean cotyledon transformation of 5 day old soybean seedling explants. Data are mean±standard error (SE) from at least three independent experiments.

FIG. 3D: Soybean embryogenic suspension cultures 5 days after transformation. Data are mean±standard error (SE) from at least three independent experiments.

FIGS. 4A-4F: Embryogenic suspension culture tissue of soybean, 5 days after transformation; 2 day co-culture and 3 day recovery a) EHA105, b) C58, c) DSOH, d) EROH, e) JTND, f) KFOH.

FIGS. 5A-5B: Typical growth of isolated novel strains on 1A semi-selective medium with tellurite added. a) A soil extract solution at 10⁻² dilution. b) A gall extract suspension at 10⁻² dilution.

FIGS. 6A-6B: After testing positive for virG, colonies are picked and streaked a minimum of three successive times on 1A medium (a), then streaked on YEP (b).

FIGS. 7A-7B: pCAMBIA1300 Gmubi3 plasmid map with (a) and without (b) splice sites.

FIGS. 8A-8G: Soybean 5 day old seedling transformation assay, after 2 day co-culture and 3 day post co-culture. Hypocotyl tissue in top images; cotyledon tissues in bottom images. a) EHA105, b) C58, c) K599, d) DSOH, e) EROH, f) JTND, g) KFOH.

### DETAILED DESCRIPTION

Described herein are materials, methods, and kits useful for Agrobacterium-mediated transformation of plant cells and plants. In particular, the present disclosure provides a novel strain of Agrobacterium and its disarmed variant. The present disclosure further provides methods and kits for transforming plant cells and plants utilizing the novel strains of Agrobacterium.

Throughout this disclosure, various publications, patents and published patent specifications are referenced. The disclosures of these publications, patents and published patent specifications are hereby incorporated by reference into the present disclosure to more fully describe the state of the art to which this invention pertains.

It is to be understood that this disclosure is not limited to the particular methodology, protocols, plant species or genera, constructs, and reagents described as such. It is also to be understood that the terminology used herein is for the purpose of describing particular embodiments only, and is not intended to limit the scope of the present invention. While a number of exemplary aspects and embodiments are discussed below, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope.

### DEFINITIONS

As used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20%, preferably 10%, up or down (higher or lower).

As used herein, the word “or” means any one member of a particular list and also includes any combination of members of that list.

As used herein, “agronomically valuable trait” includes any phenotype in a plant organism that is useful or advantageous for food or feed production or food or feed products, including plant parts and plant products. Non-food agricultural products such as paper, fiber, biofuel, or multi-use crops (such as sugarcane), etc. are also included. A partial list of agronomically valuable traits includes, but is not limited to, herbicide resistance, pest resistance, vigor, development time (i.e., time to harvest), enhanced nutrient content, novel growth patterns, flavors or colors, salt, heat, drought and cold tolerance, yield, and the like. Preferably, agronomically valuable traits do not include selectable marker genes (e.g., genes encoding herbicide or antibiotic resistance used only to facilitate detection or selection of transformed cells), hormone biosynthesis genes leading to the production of a plant hormone (e.g., auxins, gibberllins, cytokinins, abscisic acid and ethylene that are used only for selection), or reporter genes (e.g., green fluorescent protein (GFP), luciferase, glucuronidase, etc.). There are numerous polynucleotides from which to choose in order to confer these and other agronomically valuable traits.

The term “gene” refers to a nucleic acid coding region of the genome, operably joined to appropriate regulatory sequences capable of regulating the expression of a polypeptide in some manner. A gene includes untranslated regulatory regions of DNA (e.g., promoters, enhancers, repressors, etc.) up-stream and downstream of the coding region (open reading frame, ORF) as well as, where applicable, introns up-stream or between individual coding regions (i.e., exons). The term “gene” also refers to regions of the genome that provide for modification of another native gene's expression or function. Such genes are referred to as “regulatory genes,” which can be either a nucleic acid coding region, wherein the regulator gene encodes a polypeptide capable of modulating another gene's expression (e.g., transcription factor, repressor, and activator proteins), or a non-coding region, wherein the regulatory gene encodes an RNA product capable of modulating another gene's expression (e.g., anti-sense RNAs, double-stranded RNAs, microRNAs and small interfering RNAs).

As used herein the term “coding region” refers to the nucleic acid sequences which encode the amino acids found in a nascent polypeptide as a result of translation of an mRNA molecule. The coding region is bounded, in eukaryotes, on the 5′-side by the nucleotide triplet “ATG” which encodes the initiator methionine and on the 3′-side by one of the three triplets, which specify stop codons (i.e., TAA, TAG, and TGA). In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers, which control or influence the transcription of the gene. The 3′-flanking region may contain sequences, which direct the termination of transcription, post-transcriptional cleavage and polyadenylation.

As used herein the term “non-coding region” refers to a gene or nucleic acid sequence that encodes an RNA product, such as anti-sense RNA, double stranded RNA, small interfering RNAs and micro RNAs.

The term “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably, the terms genome or genomic DNA are referring to the chromosomal DNA of the nucleus.

The term “chromosomal DNA” or “chromosomal DNA-sequence” is to be understood as the genomic DNA of the cellular nucleus independent from the cell cycle status. Chromosomal DNA might therefore be organized in chromosomes or chromatids; they might be condensed or uncoiled. An insertion into the chromosomal DNA can be demonstrated and analyzed by various methods known in the art (e.g., polymerase chain reaction (PCR) analysis, Southern blot analysis, fluorescence in situ hybridization (FISH), and in situ PCR).

The term “transgenic” or “recombinant” as used herein (e.g., with regard to a plant cell or plant) is intended to refer to cells and/or plants which contains a transgene, or whose genome has been altered by the introduction of a transgene, or that have incorporated exogenous genes or DNA sequences, including but not limited to genes or DNA sequences which are perhaps not normally present, genes not normally transcribed and translated (“expressed”) in a given cell type, or any other genes or DNA sequences which one desires to introduce into the non-transformed cell and/or plant, such as genes which may normally be present in the non-transformed cell and/or plant but which one desires to have altered expression. Preferably, the term “recombinant” with respect to nucleic acids as used herein means that the nucleic acid is covalently joined and adjacent to a nucleic acid to which it is not adjacent in its natural environment. Transgenic cells, tissues and plants may be produced by several methods including the introduction of a “transgene” comprising nucleic acid (usually DNA) into a target cell or integration of the transgene into a chromosome of a target cell by way of human intervention, such as by the methods described herein.

The terms “transgene,” “transgenic,” and “recombinant” as used herein refer to a nucleic acid sequence (e.g., gene or regulatory gene) which is manipulated by human intervention or a copy or complement of a manipulated nucleic acid sequence. A transgene may be introduced into the genome of a cell by experimental manipulations. A transgene may be an “endogenous DNA sequence,” a modified endogenous DNA sequence, or a “heterologous DNA sequence” (i.e., “foreign DNA”). The term “endogenous DNA sequence” refers to a nucleotide sequence which is naturally found in the cell into which it is introduced so long as it does not contain some modification (e.g., a point mutation, the presence of a selectable marker gene, etc.) relative to the naturally-occurring sequence. Endogenous DNA sequences can be modified thereby generating a transgene, by a number of methods including for example, Zinc-finger nucleases (ZFNs), transcription activator-like effector nucleases (TALENs), and clustered regularly interspaced short palindromic repeat (CRISPR)/Cas-based RNA-guided DNA endonucleases.

The terms “transgenic” and “recombinant” as used herein (e.g., with regard to a plant cell or plant) are intended to refer to cells and/or plants which contains a transgene, or whose genome has been altered by the introduction of a transgene, or that have incorporated exogenous genes or DNA sequences. Preferably, the term “recombinant” with respect to nucleic acids as used herein means that the nucleic acid is covalently joined and adjacent to a nucleic acid to which it is not adjacent in its natural environment. Transgenic cells, tissues and plants may be produced by several methods including the introduction of a “transgene” comprising nucleic acid (usually DNA) into a target cell or integration of the transgene into a chromosome of a target cell by way of human intervention, such as by the strains of Agrobacterium and related methods described herein.

As used herein the terms “homology” and “identity” describe the extent to which sequences of DNA or protein segments are invariant throughout a window of alignment of sequences, for example nucleotide sequences or amino acid sequences. Homology and identity are calculated over the aligned length preferably using a local alignment algorithm, such as BLASTp.

For example, a sequence X with at least 95% homology (or identity) to a nucleic acid sequence Y is understood as meaning the sequence which, upon comparison with the sequence Y by a given algorithm, has at least 95% homology. There may be partial homology (i.e., partial identity of less than 100%) or complete homology (i.e., complete identity of 100%).

When used in reference to a double-stranded nucleic acid sequence such as a cDNA or genomic clone, the term “substantially homologous” refers to any probe which can hybridize to either or both strands of the double-stranded nucleic acid sequence under conditions of low stringency. When used in reference to a single-stranded nucleic acid sequence, the term “substantially homologous” refers to any probe which can hybridize to the single-stranded nucleic acid sequence under conditions of low stringency. A substantially homologous sequence or probe (i.e., an oligonucleotide which is capable of hybridizing to another oligonucleotide of interest) will compete for and inhibit the binding (i.e., the hybridization) of a completely homologous sequence to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The term “hybridization” as used herein includes any process by which a strand of nucleic acid joins with a complementary strand through base pairing. Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids.

The term “transformation” as used herein refers to the introduction and integration of one or more transgenes into the genome of a cell, preferably resulting in chromosomal integration and stable heritability through meiosis. Transformation of a cell may be detected by Southern blot hybridization of genomic DNA of the cell with nucleic acid sequences which are capable of binding to one or more of the transgenes. Alternatively, transformation of a cell may also be detected by the polymerase chain reaction of genomic DNA of the cell to amplify transgene sequences. Transformation also includes introduction of genetic material into plant cells in the form of plant viral vectors involving epichromosomal replication and gene expression which may exhibit variable properties with respect to meiotic stability.

The term “Agrobacterium” as used herein refers to a soil-borne, Gram-negative, rod-shaped phytopathogenic bacterium. The cells are normally rod-shaped (0.6-1.0 μm by 1.5-3.0 μm), occur singly or in pairs, without endospore, and are motile by one to six peritrichous flagella. Considerable extracellular polysaccharide slime is usually produced during growth on carbohydrate-containing media. The species of Agrobacterium, A. tumefaciens (syn. A. radiobacter), A. rhizogenes, A. rubi and A. vitis, together with Allorhizobium undicola, form a monophyletic group with all Rhizobium species, based on comparative 16S rDNA analyses. Agrobacterium is an artificial genus comprising plant-pathogenic species. The monophyletic nature of Agrobacterium, Allorhizobium and Rhizobium and their common phenotypic generic circumscription support their amalgamation into a single genus, Rhizobium. The classification and characterization of Agrobacterium strains including differentiation of A. tumefaciens and A. rhizogenes and their various opine-type classes is a practice well known in the art (see, for example, Laboratory guide for identification of plant pathogenic bacteria, 3rd edition. (2001) N. W. Schaad, J. B. Jones, and W. Chun (eds.) ISBN 0890542635; for example, the article of Moore et al. published therein).

Recent analyses demonstrate that classification by its plant-pathogenic properties is not justified. Accordingly more advanced methods based on genome analysis and comparison (such as 16S rRNA sequencing; RFLP, Rep-PCR, etc.) are employed to elucidate the relationship of the various strains. Agrobacteria can be differentiated into at least three biovars, corresponding to species divisions based on differential biochemical and physiological tests. Pathogenic strains of Agrobacterium share a common feature; they contain at least one large plasmid, the tumor- or root-inducing (Ti- and Ri-, respectively) plasmid. Virulence is determined by different regions of the plasmid including the transferred DNA (T-DNA) and the virulence (vir) genes. The virulence genes mediate transfer of T-DNA into infected plant cells, where it integrates into the plant DNA. According to the “traditional” classification, Agrobacteria include, but are not limited to, strains of Agrobacterium tumefaciens, (which by its natural, “armed” Ti-plasmid typically causes crown gall in infected plants), and Agrobacterium rhizogenes (which by its natural, “armed” Ri-plasmid causes hairy root disease in infected host plants), Agrobacterium rubi (which in its natural, “armed” form causes cane gall on Rubus), Agrobacterium vitis, and Agrobacterium radiobacter.

The term “Ti-plasmid” as used herein is referring to a plasmid which is replicable in Agrobacterium and is in its natural, “armed” form mediating crown gall in Agrobacterium infected plants. Infection of a plant cell with a natural, “armed” form of a Ti-plasmid of Agrobacterium generally results in the production of opines (e.g., nopaline, agropine, octopine etc.) by the infected cell. Thus, Agrobacterium strains which cause production of nopaline (e.g., strain LBA4301, C58, A208) are referred to as “nopaline-type” Agrobacteria; Agrobacterium strains which cause production of octopine (e.g., strain LBA4404, Ach5, B6) are referred to as “octopine-type” Agrobacteria; and Agrobacterium strains which cause production of agropine (e.g., strain EHA105, EHA101, A281) are referred to as “agropine-type” Agrobacteria.

A “disarmed” Ti-plasmid is understood as a Ti-plasmid lacking its crown gall mediating properties but otherwise providing the functions for plant infection. Preferably, the T-DNA region of said “disarmed” plasmid is modified in a way that besides the border sequences, no functional internal Ti-sequences can be transferred into the plant genome. In a preferred embodiment—when used with a binary vector system—the entire T-DNA region (including the T-DNA borders) is deleted, other than a gene of interest in a transgenic Ti-plasmid. A “disarmed Agrobacterium” is understood as an Agrobacterium containing a disarmed Ti-plasmid.

The term “Ri-plasmid” as used herein is referring to a plasmid, which is replicable in Agrobacterium and is in its natural, “armed” form mediating hairy-root disease in Agrobacterium infected plants. Infection of a plant cell with a natural, “armed” form of an Ri-plasmid of Agrobacterium generally results in the production of opines (specific amino sugar derivatives produced in transformed plant cells such as e.g., agropine, cucumopine, octopine, mikimopine etc.) by the infected cell. Agrobacterium rhizogenes strains are traditionally distinguished into subclasses in the same way Agrobacterium tumefaciens strains are. The most common strains are agropine-type strains (e.g., characterized by the Ri-plasmid pRi-A4), mannopine-type strains (e.g., characterized by the Ri-plasmid pRi8196) and cucumopine-type strains (e.g., characterized by the Ri-plasmid pRi2659). Some other strains are of the mikimopine-type (e.g., characterized by the Ri-plasmid pRi1724).

A disarmed Ri-plasmid is understood as a Ri-plasmid lacking its hairy-root disease mediating properties but otherwise providing the functions for plant infection. Preferably, the T-DNA region of said “disarmed” Ri plasmid was modified in a way, that beside the border sequences, no functional internal Ri-sequences can be transferred into the plant genome. In a preferred embodiment—when used with a binary vector system—the entire T-DNA region (including the T-DNA borders) is deleted, other than a gene of interest in a transgenic Ri-plasmid.

As used herein the term “substantially biologically pure” means that a culture fluid, culture plate, or other collection of materials (e.g., bacteria, DNA, RNA, plasmid) is homogenous or uniformly of a single form of the material (e.g., single strain of bacteria, DNA, RNA, or plasmid), with greater that 90% purity, preferably at least 95% pure, and more preferably at least 98%.

The terms “polypeptide”, “peptide”, “oligopeptide”, “polypeptide”, “gene product”, “expression product” and “protein” are used interchangeably herein to refer to a polymer or oligomer of consecutive amino acid residues.

“Recombinant” polypeptides or proteins refer to polypeptides or proteins produced by recombinant DNA techniques, i.e., produced from cells transformed by an exogenous recombinant DNA construct encoding the desired polypeptide or protein. Recombinant nucleic acids and polypeptide may also comprise molecules, which as such does not exist in nature but are modified, changed, mutated or otherwise manipulated by man. A “recombinant polypeptide” may be a non-naturally occurring polypeptide that differs in sequence from a naturally occurring polypeptide by at least one amino acid residue. Preferred methods for producing said recombinant polypeptide and/or nucleic acid may comprise directed or non-directed mutagenesis, DNA shuffling or other methods of recursive recombination.

As used herein, the term “expression” refers to the biosynthesis of a gene product. For example, expression involves transcription of a gene into mRNA and—optionally—additional elements which facilitate expression of the nucleic acid sequence.

The terms “expression cassette” and “expression construct” as used herein are intended to mean the combination of any nucleic acid sequence to be expressed in operable linkage with a promoter sequence and—optionally—additional elements (like e.g., terminator and/or polyadenylation sequences) which facilitate expression of said nucleic acid sequence.

As used herein, the terms “promoter,” “promoter element,” or “promoter sequence” refer to a DNA sequence which when ligated to a nucleotide sequence of interest is capable of controlling the transcription of the nucleotide sequence of interest in a plant. A promoter is typically, though not necessarily, located upstream of a nucleotide sequence of interest (e.g., proximal to the transcriptional start site of a structural gene) whose transcription into mRNA it controls, and provides a site for specific binding by RNA polymerase and other transcription factors for initiation of transcription. A polynucleotide sequence is “heterologous” to an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from plants or plant pathogens like e.g., plant viruses.

If a promoter is an inducible promoter, then the rate of transcription increases in response to an inducing agent. In contrast, the rate of transcription is not regulated by an inducing agent if the promoter is a constitutive promoter. Also, the promoter may be regulated in a tissue-specific or tissue preferred manner such that it is only active in transcribing the associated coding region in a specific tissue type(s) such as leaves, roots, or meristem. The term “tissue specific” as it applies to a promoter refers to a promoter that is capable of directing selective expression of a nucleotide sequence of interest to a specific type of tissue (e.g., petals) in the relative absence of expression of the same nucleotide sequence of interest in a different type of tissue (e.g., roots). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected.

The term “cell type specific” as applied to a promoter refers to a promoter which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using, for example, GUS activity staining and immunohistochemical staining.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. In contrast, a “regulatable” promoter is one which is capable of directing a level of transcription of an operably linked nucleic acid sequence in the presence of a stimulus (e.g., heat shock, chemicals, light, etc.) which is different from the level of transcription of the operably linked nucleic acid sequence in the absence of the stimulus.

Where expression of a gene in all tissues of a transgenic plant or other organism is desired, one can use a “constitutive” promoter, which is generally active under most environmental conditions and states of development or cell differentiation. Useful promoters for plants also include those obtained from Ti- or Ri-plasmids, from plant cells, plant viruses, or other organisms whose promoters are found to be functional in plants. Bacterial promoters that function in plants, and thus are suitable for use in the methods of the invention include the octopine synthetase promoter, the nopaline synthase promoter, and the mannopine synthetase promoter. Suitable constitutive promoters for use in plants include, for example, the cauliflower mosaic virus (CaMV) 35S transcription initiation region, the Gmubi promoter from soybean, the elongation factor 1 alpha promoter from soybean, and other promoters active in plant cells.

One can use a promoter that directs expression of a gene of interest in a specific tissue or is otherwise under more precise environmental or developmental control. Examples of environmental conditions that may affect transcription by inducible promoters include pathogen attack, anaerobic conditions, ethylene, or the presence of light. Promoters under developmental control include promoters that initiate transcription only in certain tissues or organs, such as leaves, roots, fruit, seeds, or flowers, or parts thereof. The operation of a promoter may also vary depending on its location in the genome. Thus, an inducible promoter may become fully or partially constitutive in certain locations. Examples of tissue-specific plant promoters under developmental control include promoters that initiate transcription only in certain tissues, such as fruit, seeds, flowers, anthers, ovaries, pollen, the meristem, flowers, leaves, stems, roots and seeds. The tissue-specific ES promoter from tomato is particularly useful for directing gene expression so that a desired gene product is located in fruits. Other suitable seed-specific promoters are known in the art.

An expression cassette may also contain a chemically inducible promoter, by means of which the expression of the exogenous gene in the plant can be controlled at a particular point in time. Such promoters such as, for example, the PRP1 promoter, and a salicylic acid-inducible promoter, among many others. A promoter that responds to an inducing agent to which plants do not normally respond can be utilized. Other preferred promoters are promoters induced by biotic or abiotic stress, such as, for example, the pathogen-inducible promoter of the PRP1 gene, the tomato heat-inducible hsp80 promoter, the potato chill-inducible alpha-amylase promoter, and the wound-induced pinII promoter, among others.

As used herein “operably linked” is to be understood as meaning, for example, the sequential arrangement of a regulatory element (e.g. a promoter) with a nucleic acid sequence to be expressed and, if appropriate, further regulatory elements (such as e.g., a terminator) in such a way that each of the regulatory elements can fulfill its intended function to allow, modify, facilitate or otherwise influence expression of said nucleic acid sequence. The expression may result depending on the arrangement of the nucleic acid sequences in relation to sense or antisense RNA. To this end, direct linkage in the chemical sense is not necessarily required. Genetic control sequences such as enhancer sequences can also exert their function on the target sequence from positions which are further away or from other DNA molecules. Preferred arrangements are those in which the nucleic acid sequence to be expressed recombinantly is positioned downstream of the sequence acting as promoter, so that the two sequences are linked covalently to each other. Operable linkage, and an expression construct, can be generated by means of customary recombination and cloning techniques. However, further sequences which act as a linker with specific cleavage sites for restriction enzymes, or as a signal peptide, may also be positioned between the two sequences. The insertion of sequences may also lead to the expression of fusion proteins. Preferably, the expression construct, consisting of a linkage of promoter and nucleic acid sequence to be expressed, can exist in a vector-integrated form and be inserted into a plant genome, for example, by transformation.

The terms “infecting” and “infection” with a bacterium refer to co-culture of a target biological sample (e.g., cell, tissue, etc.) with the bacterium under conditions such that nucleic acid sequences contained within the bacterium are introduced into one or more cells of the target biological sample.

The term “isolated” as used herein means that a material has been removed from its original environment. For example, a naturally occurring polynucleotide or polypeptide present in a living animal is not isolated, but the same polynucleotide or polypeptide, separated from some or all of the coexisting materials in the natural system, is isolated. Such polynucleotides can be part of a vector and/or such polynucleotides or polypeptides could be part of a composition, and would be isolated in that such a vector or composition is not part of its original environment. Preferably, the term “isolated” when used in relation to a nucleic acid, as in “an isolated nucleic acid sequence” refers to a nucleic acid sequence that is identified and separated from at least one contaminant nucleic acid with which it is ordinarily associated in its natural system. Isolated nucleic acid is nucleic acid present in a form or setting that is different from that in which it is found in nature. In contrast, non-isolated nucleic acids are nucleic acids such as DNA and RNA which are found in the state they exist in nature. An isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

The term “tissue” with respect to a plant (or “plant tissue”) means arrangement of multiple plant cells including differentiated and undifferentiated tissues of plants. Plant tissues may constitute part of a plant organ (e.g., the epidermis of a plant leaf) but may also constitute tumor tissues (e.g., callus tissue) and various types of cells in culture (e.g., single cells, protoplasts, embryos, calli, protocorm-like bodies, etc.). Plant tissue may be in planta, in organ culture, tissue culture, or cell culture.

The term “plant” as used herein refers to a plurality of plant cells, which are largely differentiated into a structure that is present at any stage of a plant's development. Such structures include one or more plant organs including, but are not limited to, fruit, shoot, stem, leaf, flower petal, etc. Preferably, the term “plant” includes whole plants, shoot vegetative organs/structures (e.g. leaves, stems and tubers), roots, flowers and floral organs/structures (e.g. bracts, sepals, petals, stamens, carpels, anthers, and ovules), seeds (including embryo, endosperm, and seed coat), fruits (the mature ovary), plant tissues (e.g. vascular tissue, ground tissue, and the like), cells (e.g. guard cells, egg cells, trichomes and the like), and progeny of same. The class of plants that can be used in the methods of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous. Included within the scope of the invention are all genera and species of higher and lower plants of the plant kingdom. Included are furthermore the mature plants, seed, shoots and seedlings, and parts, propagation material (for example seeds and fruit), and cultures (e.g., cell cultures) derived therefrom.

Annual, perennial, gymnosperms, monocotyledonous and dicotyledonous plants are preferred host organisms for the generation of transgenic plants. The use of Agrobacterium strains and methods according to the invention is furthermore advantageous in crop plants, ornamental plants, forestry, fruit, ornamental trees, flowers, cut flowers, shrubs, and turf.

Plants useful for the purposes of the present disclosure may comprise for example, the Fabaceae family, such as pea, alfalfa and soybean; the Umbelliferae family, particularly the genus Daucus (very particularly the species carota (carrot)) and Apium (very particularly the species graveolens var. dulce (celery)); the Solanaceae family, particularly the genus Lycopersicon, very particularly the species esculentum (tomato) and the genus Solanum, very particularly the species tuberosum (potato) and melongena (aubergine); the Cruciferae family, particularly the genus Brassica, very particularly the species napus (oilseed rape), campestris (beet), oleracea cv Tastie (cabbage), oleracea cv Snowball Y (cauliflower) and oleracea cv Emperor (broccoli); and the Compositae family, particularly the genus Lactuca, very particularly the species sativa (lettuce). Plants useful for the purposes of the present disclosure may comprise for example, plants of the genera Glycine; Medicago; Pisum; Beta, Helianthus; Arabidopsis; Dioscorea; Ipomea; Manihot; Plantago; Zea; Oryza; Sorghum; Triticum; Hordeum; Saccharum; Brassica; Solanum; Nicotiana; Gossypium; Vitis; Populus; Picea; and Pinus.

The transgenic plants according to the invention are selected in particular among monocotyledonous crop plants, such as, for example, cereals such as wheat, barley, sorghum, millet, rye, triticale, maize, rice or oats, and sugarcane. Further preferred are trees such as apple, pear, quince, plum, cherry, peach, nectarine, apricot, papaya, mango, and other woody species including coniferous and deciduous trees such as poplar, pine, sequoia, cedar, oak, etc. Also preferred are Arabidopsis thaliana, Nicotiana tabacum, oilseed rape, soybean, corn (maize), wheat, linseed, potato and Tagetes.

General Description

Described herein is Agrobacterium strain JTND, capable of transforming plant cells, plant tissues, and plants. Also described herein is Agrobacterium strain SBHT, a disarmed strain variant of JTND also capable of transforming plant cells, plant tissues, and plants. In particular embodiments, Agrobacterium strains JTND and SBHT can be used to transform virtually all types of plants. Preferred are plant cells or plant tissues derived from plants, or plants, selected from the group of monocotyledonous plants, dicotyledonous plants, and gymnosperm plants. More preferably, the plant cells or plant tissues derived from plants, or plants, selected from genus selected from the group consisting of Glycine, Medicago, Pisum, Beta, Helianthus, Arabidopsis, Dioscorea, Ipomea, Manihot, Plantago, Zea, Oryza, Sorghum, Triticum, Hordeum, Saccharum, Brassica, Solanum, Nicotiana, Gossypium, Vitis, Populus, Picea, and Pinus. Most preferably, the plant cells or plant tissues derived from plants, or plants are Glycine max (soybean). Agrobacterium strains JTND and SBHT shall be deposited with the American Type Culture Collection (ATCC). ATCC accession numbers will be provided following deposit (see Deposit Information section below).

In certain embodiments, Agrobacterium strains described herein comprise one or more mutant or chimeric virA gene, virG gene, or super-virulent plasmids. In other embodiments, Agrobacterium strains described herein are capable of transforming plant cells, plant tissues, and plants, but are lacking tumor inducing properties. In yet other embodiments, Agrobacterium strains described herein further comprise transgenic T-DNA.

The transgenic T-DNA may contain one or more selectable marker genes suitable for selection. Selectable marker genes are useful to select and separate successfully transformed or homologous recombined plant cells, plant tissues, or plants. Preferably, within the methods described herein, one marker may be employed for selection in a prokaryotic host, while another marker may be employed for selection in a eukaryotic host, particularly the plant species host. The markers may be protection against a biocide, such as antibiotics, toxins, heavy metals, or the like, or may function by complementation, imparting prototrophy to an auxotrophic host. Selectable markers may be, but are not limited to, negative selection markers, positive selection markers, and counter selection markers.

Negative selection markers confer a resistance to a biocidal compound such as a metabolic inhibitor (e.g., 2-deoxyglucose-6-phosphate), antibiotics (e.g., kanamycin, G 418, bleomycin, or hygromycin), or herbicides (e.g., phosphinothricin or glyphosate). Preferred negative selection markers are those which confer resistance to antibiotics. Especially preferred negative selection markers are those which confer resistance to hygromycin (e.g., hygromycin phosphotransferase).

Positive selection markers are those that confer a growth advantage to a transformed plant in comparison with a non-transformed plant. Genes like isopentenyltransferase from Agrobacterium tumefaciens (strain: PO22; Genbank Acc.-No.: AB025109) may facilitate regeneration of transformed plants (e.g., by selection on cytokinin-free medium). Growth stimulation selection markers may include (but shall not be limited to) β-Glucuronidase (in combination with e.g., cytokinin glucuronide), mannose-6-phosphate isomerase (in combination with mannose), and UDP-galactose-4-epimerase (in combination with e.g., galactose).

Counter selection markers are suitable to select organisms with defined deleted sequences comprising the marker. Examples for negative selection marker comprise thymidine kinases (TK), cytosine deaminases, cytochrome P450 proteins, haloalkane dehalogenases, iaaH gene products, cytosine deaminase codA, and tms2 gene products.

In particular embodiments, transgenic T-DNA may contain one or more reporter genes. Reporter genes encode readily quantifiable proteins and, via their color or enzyme activity, make possible an assessment of the transformation efficacy, the site of expression, and/or the time of expression. Preferred in this context are genes encoding reporter proteins such as green fluorescent protein (GFP), chloramphenicol acetyltransferase, a luciferase, the aequorin gene, R locus gene (encoding a protein which regulates the production of anthocyanin pigments (red coloring) in plant tissue and thus makes possible the direct analysis of the promoter activity without addition of further auxiliary substances or chromogenic substrates), and β-glucuronidase (GUS). β-glucuronidase (GUS) expression is detected by a blue color on incubation of the tissue with 5-bromo-4-chloro-3-indolyl-β-D-glucuronic acid, bacterial luciferase (LUX) expression is detected by light emission; firefly luciferase (LUC) expression is detected by light emission after incubation with luciferin. Particularly preferred is the use of GFP, which may be directly observed and analyzed utilizing fluorescence microscopy. Reporter genes may also be used as scorable markers as alternatives to antibiotic resistance markers. Such markers are used to detect the presence or to measure the level of expression of the transferred gene. The use of scorable markers in plants to identify or tag genetically modified cells works well only when efficiency of modification of the cell is high.

In a preferred embodiment, transgenic T-DNA contains a gene or regulatory gene of interest. A gene of interest is a gene one wishes to incorporate into the genome of a plant cell, plant tissue, or plant by means of the methods described herein (e.g., Agrobacterium-mediated transformation). A gene of interest is preferably one that is associated with an agronomically valuable trait, and confers the agronomically valuable trait to a transformed plant cell, plant tissue, or plant. Agronomically valuable traits are discussed above. Regulatory genes of interest include nucleic acids that encode, for example, anti-sense RNA, double-stranded RNA, small interfering RNA, and micro RNA. Regulatory genes of interest are preferably those capable of affecting the expression of other native genes. Expression of other native genes by a regulatory gene may be by way of transcription factors capable of binding DNA regulatory regions leading to transcription of DNA into RNA, repressor proteins capable of binding operators or promoters of a native gene, thereby preventing transcription, activator proteins capable of increasing transcription of a native gene, or RNA products capable of affecting native gene expression (e.g., anti-sense RNA, double-stranded RNA, small interfering RNA, and micro RNA). A regulatory gene of interest is preferably one that regulates a native gene, wherein the native gene itself is associated an agronomically valuable trait to a plant.

In another preferred embodiment, the Agrobacterium strains JTND and SBHT are capable of transforming plant cells, plant tissues, and plants, by mediating T-DNA transfer into the plant genome. In especially preferred embodiments, the Agrobacterium strain lacks tumor inducing properties. The Agrobacterium strain provides all functions required for plant cell infection and transformation but is lacking tumor inducing DNA sequences.

In other embodiments, the Agrobacterium strains JTND and SBHT are capable of introducing genome editing nucleases into a plant cell, plant tissue, or plant. In such embodiments, transgenic T-DNA contains one or more nuclease-encoding genes. Expressed in the plant cell, plant tissue, or plant, the genome editing nucleases aid in the insertion of a gene of interest or regulatory gene of interest, replace a native gene with a gene of interest or regulatory gene of interest, or remove or modify a native gene or native regulatory gene. Preferably, the encoded nucleases are targeted to a specific position in the plant's genome. Introducing genome editing nucleases into a plant cell, plant tissue, or plant can provide for trait stacking, resulting in the physical linkage of certain traits to ensure co-segregation during breeding. Any genome editing nucleases known in the art may be used, including but not limited to Zinc-finger nucleases (ZFNs), transcription activator-like effector nucleases (TALENs), and clustered regularly interspaced short palindromic repeat (CRISPR)/Cas-based RNA-guided DNA endonucleases.

In yet another preferred embodiment, the Agrobacterium strains JTND and SBHT are further modified to increase the transformation efficiency, such as by altering vir gene expression and/or induction thereof. This may be realized by the presence of mutant or chimeric virA or virG genes. Combinations with super-virulent plasmids are also possible, generating so-called super-virulent strains. Super-virulent strain variants may also be generated by employing pSB1 super virulence plasmid derived vectors.

Agrobacterium Strain JTND

Agrobacterium strain JTND was collected from a soil sample from a soybean field following screening of many soil samples from fields throughout the Midwest United States. The strain was isolated and identified during a search for new strains of Agrobacterium having higher transformation efficiencies in specific plants.

The “armed” JTND comprises an intact Ti-plasmid carrying an unaltered T-DNA region, as well as all vir genes. In particular embodiments, JTND may be transformed with a binary plasmid (lacking tumor inducing properties) incorporating a reporter gene (e.g., green fluorescent protein, GFP). In other embodiments, a binary plasmid comprises one or more genes of interest, regulatory genes of interest, selectable marker genes, reporter genes, or combinations thereof.

Preferably, the T-DNA of a binary vector to be incorporated in JTND comprises a transgenic T-DNA region flanked by at least one T-DNA border sequence, but comprises no tumor inducing sequences.

Preferably, the T-DNA of the binary vector is flanked by at least the right border sequence, and more preferably, by both the right and left border sequences. T-DNA border sequences are repeats of about 25 bp, and are well described and defined in the art. The borders mediate T-DNA transfer, in conjunction with vir genes found on the transgenic Ti plasmid (lacking T-DNA).

Preferably, the binary vector comprises no sequences that result in a tumor phenotype, more preferably the plasmid comprises no internal T-DNA protein-encoding sequences, most preferably the plasmid comprises substantially no internal T-DNA sequences. The term “internal” in this context means the DNA flanked by, but excluding the T-DNA borders. The term “substantially” is intended to mean that some internal sequences which are not linked to a pathogenic phenotype may be included. Preferably, these sequences are not more than 200 bp, preferably not more than 100 bp, most preferably not more than 50 bp, and are preferably directly consecutive to the border sequences.

The T-DNA to be incorporated into the plant genome by the JTND strain can be provided in various forms. The T-DNA may be provided as a DNA construct, preferably integrated into specific vectors, either into a shuttle or intermediate vector, or into a binary vector. The T-DNA may be incorporated in the Ti plasmid of the JTND strain, or may be incorporated in the JTND strain in the form of a binary vector separate from JTND's Ti plasmid. In another preferred embodiment, the T-DNA of the binary vector comprises one or more genes of interest, regulatory genes of interest, marker genes, reporter genes, or combinations thereof.

Binary vectors described herein may be transferred into Agrobacterium strain JTND for example by electroporation, the freeze-thaw method, particle bombardment, or other transformation methods. Binary vectors described herein are capable of replication in both E. coli and in Agrobacterium. Binary vectors can be transformed directly into Agrobacterium, which should already have a Ti plasmid with an intact vir region. The Agrobacterium strain JTND, thus transformed, can be used for transforming plant cells, plant tissues, and plants.

Disarmed Agrobacterium strain SBHT

It is preferable that strains of Agrobacterium to be used with binary vectors have its own Ti plasmid disarmed, especially if the target plant species is inefficiently transformed by Agrobacterium. Otherwise, the gene(s) of interest or regulatory gene(s) of interest from the binary vector will be co-transformed along with the tumor-inducing genes from the native T-DNA of the bacteria, reducing transformation efficiency of the target gene(s) and also producing tumorigenic disease symptoms in many of the target cells, preventing differentiation of these cells into normal plants

JTND can be disarmed to produce a disarmed derivative strain by several means, including but not limited to: 1) rendering the left and right borders of the T-DNA dysfunctional; 2) deleting the entire native T-DNA region from the Ti plasmid; 3) deletion mutagenesis by inducing DNA nicks and excision of the T-DNA; 4) transposon mutagenesis and screening for a non-pathogenic mutant; and 5) directed and specific deletion of relevant genes (by replacing wild-type copies of genes between the right border and left border with a deleted replacement, only the genes that need to be deleted are excised). In a particular embodiment, disarmed Agrobacterium strain SBHT was developed by deleting the entire native T-DNA region from the Ti plasmid of the JTND strain.

The disarmed SBHT has a Ti-plasmid lacking the entire native T-DNA region, while the vir genes remain on the Ti-plasmid. Preferably, the T-DNA of disarmed Agrobacterium strain SBHT comprises a binary vector separate from the disarmed Ti plasmid (lacking T-DNA). With the entire native T-DNA region removed, as in SBHT, a binary vector system is used to transform plant cells, plant tissues, and plants. In a binary vector system, binary vectors are small T-DNA vectors with a cloning site and a transgene located between the left and right border sequences. The transgene can be a gene of interest, a regulatory gene (e.g., miRNA, siRNA), a marker gene, a reporter gene, or combinations thereof. In particular embodiments, the binary vector comprises, besides the disarmed T-DNA with its border sequences, prokaryotic sequences for replication both in Agrobacterium and E. coli.

Preferably, the T-DNA is flanked by at least the right border sequence, and more preferably, by both the right and left border sequences. T-DNA border sequences are repeats of about 25 bp, and are well described and defined in the art. The borders mediate T-DNA transfer, in conjunction with vir genes found on the transgenic Ti plasmid (lacking native T-DNA).

Preferably, the binary vector comprises no sequences that result in a tumor phenotype, more preferably the plasmid comprises no internal T-DNA protein-encoding sequences, most preferably the plasmid comprises substantially no internal T-DNA sequences. The term “internal” in this context means the DNA flanked by, but excluding the T-DNA borders. The term “substantially” is intended to mean that some internal sequences which are not linked to a pathogenic phenotype may be included. Preferably, these sequences are not more than 200 bp, preferably not more than 100 bp, most preferably not more than 50 bp, and are preferably directly consecutive to the border sequences.

The T-DNA to be incorporated into the plant genome by the disarmed SBHT strain can be provided in various forms. The T-DNA can be provided as a DNA construct, preferably integrated into specific vectors, either into a shuttle or intermediate vector, or into a binary vector. The T-DNA can be incorporated in the disarmed Ti plasmid of the disarmed SBHT strain, or can be incorporated in the disarmed SBHT strain in the form of a binary vector separate from the disarmed Ti plasmid. The T-DNA in the disarmed SBHT strain is incorporated in a binary vector separate from the disarmed Ti plasmid. In another preferred embodiment, the T-DNA of the binary vector comprises one or more genes of interest, regulatory genes, marker genes, reporter genes, or combinations thereof.

Binary vectors described herein can be transferred into disarmed Agrobacterium strain SBHT, for example, by electroporation, the freeze-thaw method, particle bombardment, or other transformation methods. Binary vectors described herein are capable of replication in both E. coli and in Agrobacterium. Binary vectors can be transformed directly into Agrobacterium, which should already contain a transgenic, disarmed Ti plasmid with the vir region intact (e.g., SBHT). The Agrobacterium strain SBHT, thus transformed, can be used for transforming plant cells, plant tissues, and plants.

Vectors

Descriptions of Agrobacterium vector systems and methods for Agrobacterium-mediated transformation are known in the art. All vectors suitable for transformation based on Agrobacterium tumefaciens can also be employed for the methods described herein. Common binary vectors are based on “broad host range” plasmids like pRK252 or pTJS75, derived from the P-type plasmid RK2. Most of these vectors are derivatives of pBIN19. Various binary vectors are known, some of which are commercially available such as, for example, pBI101.2 or pBIN19 (Clontech Laboratories, Inc. USA), and pCAMBIA1300 (Cambia, Canberra, Australia). A binary vector or any other vector can be modified by common DNA recombination techniques, multiplied in E. coli, and introduced into Agrobacterium by e.g., electroporation, the freeze-thaw method, particle bombardment, or other transformation methods

Expression Cassettes

In a preferred embodiment, the T-DNA region to be integrated into the plant genome by means of the Agrobacterium strains described herein, and more preferably the disarmed strain SBHT, comprises at least one expression cassette for conferring to said plant an agronomically valuable trait. In another preferred embodiment, the T-DNA region further comprises at least one marker gene, which allows for selection and/or identification of transformed plant cells, plant tissues, or plants. Thus, the T-DNA inserted into the genome of the target plant cell, plant tissue or plant comprises at least one expression cassette, which may facilitate expression of genes of interest, regulatory genes of interest, selectable marker genes, anti-sense RNA, double-stranded RNA, or a combination thereof. Preferably, the expression cassettes of the binary vector comprise a promoter sequence functional in plant cells which is operably linked to a nucleic acid sequence which confers an advantageous and/or agronomically valuable phenotype to the transformed plant. Such sequences may be those that result in an increase in quality of food and feed, to produce chemical or pharmaceuticals, confer resistance to herbicides, or confer male sterility, among others. Growth, yield, and resistance to abiotic (drought, nutrient availability) and biotic stress (e.g., fungi, viruses, insects) may also be enhanced by such sequences. These advantageous and/or agronomically valuable phenotypes may be achieved by overexpressing or decreasing expression of endogenous proteins, or by introducing heterologous DNA resulting in expression of a heterologous protein.

For expression in plants, plant-specific promoters are preferably incorporated in the T-DNA region. The term “plant-specific” is understood as meaning, in principle, any promoter which is capable of governing the expression of genes, in particular heterologous genes, in plants or plant parts, plant cells, plant tissues, or plant cultures. Expression may be constitutive, inducible, or development-dependent.

The genetic component and/or the expression cassette may further comprise genetic control sequences in addition to a promoter. The term “genetic control sequences” is to be understood in the broad sense and refers to all those sequences which affect the making or function of a DNA construct. For example, genetic control sequences modify the transcription and translation in prokaryotic or eukaryotic organisms. Preferably, expression cassettes encompass a promoter functional in plants 5′-upstream of the nucleic acid sequence in question to be expressed recombinantly, and 3′-downstream a terminator sequence as additional genetic control sequence and, if appropriate, further customary regulatory elements, in each case linked operably to the nucleic acid sequence to be expressed recombinantly.

Examples of such control sequences are sequences to which inductors or repressors bind and thus regulate the expression of the nucleic acid. Genetic control sequences furthermore also encompass the 5′-untranslated region, introns or the non-coding 3′-region of genes, such as, for example, the actin-1 intron, or the Adh1-S introns 1, 2 and 6. It has been demonstrated that they may play a significant role in the regulation of gene expression. Thus, it has been demonstrated that 5′-untranslated sequences are capable of enhancing the transient expression of heterologous genes. Examples of translation enhancers include the tobacco mosaic virus 5′-leader sequence and the like. Furthermore, they may promote tissue specificity. Conversely, the 5′-untranslated region of the opaque-2 gene suppresses expression. Deletion of the region leads to an increased gene activity. Genetic control sequences may also encompass ribosome-binding sequences for initiating translation. This is preferred in particular when the nucleic acid sequence to be expressed does not provide suitable sequences or when they are not compatible with the expression system.

The expression cassette may advantageously comprise one or more enhancer sequences operably linked to the promoter, which make possible an increased recombinant expression of the nucleic acid sequence. Additional advantageous sequences, such as further regulatory elements or terminators, may also be inserted at the 3′-end of the nucleic acid sequences to be expressed recombinantly.

Polyadenylation signals which are suitable as control sequences are preferably those which essentially correspond to T-DNA polyadenylation signals from Agrobacterium tumefaciens, in particular the octopine synthase (OCS) terminator and the nopaline synthase (NOS) terminator.

One or more copies of the nucleic acid sequences to be expressed recombinantly (e.g., regulatory gene of interest or gene of interest) may be present in the gene construct. Genetic control sequences are furthermore understood as meaning sequences which encode fusion proteins consisting of a signal peptide sequence.

Control sequences are furthermore to be understood as those permitting removal of the inserted sequences from the genome. Methods based on the cre/lox, or Ac/Ds system permit removal of a specific DNA sequence from the genome of the host organism. Control sequences may in this context mean the specific flanking sequences (e.g., lox sequences), which later allow removal (e.g., by means of cre recombinase).

The genetic component and/or expression cassette of the invention may comprise further functional elements. The term functional element is to be understood in the broad sense and refers to all those elements which have an effect on the generation, amplification or function of the genetic component, expression cassettes, or recombinant organisms according to the invention. Functional elements may include, but shall not be limited to, for example: selectable marker genes, including negative selection markers and counter selection markers; reporter genes; and origins of replication

Transformation Methods

Methods described herein are useful for obtaining transgenic plant cells, plant tissues, and plants, and cells, plant parts, tissues, and harvested material (e.g., seeds) harvested therefrom.

Accordingly, particular embodiments described herein relate to transgenic plant cell, plant tissues, and plants comprising in their genome, preferably in their nuclear chromosomal DNA, the DNA construct according to the invention (e.g., a JTND Ti-plasmid having heterologous DNA incorporated in the T-DNA region, a JTND Ti-plasmid along with a binary vector having heterologous DNA incorporated in the T-DNA region, a disarmed SBHT Ti-plasmid along with a binary vector having heterologous DNA incorporated in the T-DNA region), and to cells, cell cultures, tissues, parts or propagation material derived from such plants such as, for example, leaves, roots, seeds, fruit, pollen and the like. Other important aspects of the invention include the progeny of the transgenic plants prepared by the disclosed methods, as well as the cells derived from such progeny, and the seeds obtained from such progeny.

In addition to a plant, the present disclosure provides any clone of such a plant, seed, selfed or hybrid progeny and descendants, and any part or propagule of any of these, such as cuttings and seed, which may be used in reproduction or propagation, sexual or asexual. Also encompassed is a plant which is a sexually or asexually propagated offspring, clone or descendant of such a plant, or any part or propagule of said plant, offspring, clone or descendant. Genetically modified plants according to the disclosure which can be consumed by humans or animals can also be used as food or feedstuffs, for example directly or following processing known in the art.

The methods described herein can virtually be employed on all plants varieties, including varieties of monocotyledonous and dicotyledonous plants (as defined and specified above). Numerous explants, plant tissues, or plant cell culture may be employed as target material for the co-cultivation process. After a DNA construct is stably incorporated in transgenic plants and confirmed to be operable, it can be introduced into other plants by sexual crossing. Any of a number of standard breeding techniques can be used, depending upon the species to be crossed.

To transfer the DNA to the plant cell, plant tissue, or plant, plant explants are co-cultured with an Agrobacterium strain described herein comprising transgenic T-DNA. Starting from transformed plant material (for example leaf, root or stalk sections, but also protoplasts or suspensions of plant cells), intact plants can be regenerated using a suitable medium which may contain, for example, antibiotics or biocides for selecting transformed cells. The plants obtained can then be screened in the presence of the introduced DNA. As soon as the DNA has integrated into the host genome, the genotype in question is, as a rule, stable and the insertion in question are also found in the subsequent generations. Preferably, a stably transformed plant is selected utilizing a selection marker integrated in the transgenic T-DNA. The plants obtained can be cultured and hybridized in the customary fashion. Two or more generations should be grown in order to ensure that the genomic integration is stable and heritable.

Various tissues are suitable as starting material for the Agrobacterium-mediated transformation process including but not limited to pollen, ovule, immature plant embryo, mature plant embryo, seed, seedling, root, cotyledon, stem, node, internode, bud, leaf, shoot apical meristem, and cultured plant material. The methods and materials described herein can be combined with virtually all Agrobacterium-mediated transformation methods known in the art, including but not limited to incubating host plant starting material with Agrobacterium, co-cultivation of the host plant starting material and the Agrobacterium, the floral dip method, the vacuum infiltration method, the cotyledonary-node method, and sonication-assisted Agrobacterium-mediated transformation.

Efficiency of transformation with Agrobacterium can be enhanced by numerous methods known in the art like, for example, wounding, vacuum infiltration, heat shock and/or centrifugation, addition of silver nitrate, sonication etc. In a preferred embodiment, the explant material is wounded prior to inoculation (co-cultivation) with Agrobacterium. Many methods of wounding can be used, including, for example, cutting, abrading, piercing, poking, penetration with fine particles or pressurized fluids, plasma wounding, application of hyperbaric pressure, or sonication. Wounding can be performed using objects such as, but not limited to, scalpels, scissors, needles, abrasive objects, airbrush, particles, electric gene guns, or sound waves. Another alternative is vacuum infiltration. Other methods to increase Agrobacterium transformation efficiency known in the art can be combined, including but not limited to sonication of the target tissue.

The Agrobacterium strains JTND and SBHT described herein are grown and used in a manner as known in the art. A vector-comprising Agrobacterium strain may, for example, be grown for 3 days in YEP medium (see Example 1) supplemented with the appropriate antibiotic (e.g., 50 mg/L kanamycin). Bacteria may be collected by centrifugation and resuspended. In a particular embodiment, Agrobacterium cultures are started by use of aliquots frozen at −80° C. Agrobacterium may be resuspended in the medium used for culture of plant tissues.

The concentration of Agrobacterium used for infection and co-cultivation may need to be varied. Thus, a range of Agrobacterium concentrations from 10² to 10¹⁰ cfu/mL and a range of co-cultivation periods from a few hours to 14 days can be used. Plant material may be inoculated with the Agrobacterium culture for a few minutes to a few hours, typically about 10 minutes to 3 hours. The excess media is then drained and the Agrobacterium are permitted to co-cultivate with the target tissue for several days, generally carried out for 1 to 14, preferably 2 to 4 days. During this step, the Agrobacterium transfers the genes within the T-DNA into cells of the target tissue. Normally no selection agent presents during this step.

It is possible, although not necessary, to employ one or more phenolic compounds in the medium prior to or during the Agrobacterium co-cultivation. “Plant phenolic compounds” or “plant phenolics” suitable within the scope of the invention are those isolated substituted phenolic molecules which are capable to induce a positive chemotactic response, particularly those who are capable to induce increased vir gene expression in a Ti plasmid-containing strain of Agrobacterium. Preferred is acetosyringone. Moreover, certain compounds, such as osmoprotectants (e.g. L-proline preferably at a concentration of about 700 mg/L or betaine), phytohormones (inter alia NAA), opines, or sugars, are expected to act synergistically when added in combination with plant phenolic compounds. The plant phenolic compound, particularly acetosyringone, can be added to the medium prior to contacting the starting material with Agrobacterium (for e.g., several hours to one day). Possible concentrations of plant phenolic compounds in the medium range from about 25 μM to 700 μM, preferably 100-200 μM.

Supplementation of the co-cultivation medium with antioxidants (e.g., dithiothreitol, L-cysteine) which can decrease tissue necrosis due to plant defense responses (like phenolic oxidation) may further improve the efficiency of Agrobacterium-mediated transformation.

After co-cultivation, steps can be included to remove, suppress growth, or kill the Agrobacterium. These steps may include one or more washing steps. The medium employed after the co-cultivation step preferably contains an antibiotic. This step is intended to kill the remaining Agrobacterium cells. Preferred antibiotics to be employed are, for example, carbenicillin (500 mg/L) or Timentin™ (GlaxoSmithKline; a mixture of ticarcillin disodium and clavulanate potassium; 0.8 g Timentin™ contains 50 mg clavulanic acid with 750 mg ticarcillin).

After the co-cultivation step, the co-cultivated starting material is preferably incubated on a regeneration medium comprising at least one plant growth factor. The employed media may further contain at least one compound, which in combination with the selectable marker gene allows for identification and/or selection of plant cells (e.g., a selective agent) may be applied. Starting material may be incubated for a certain time (e.g., 5 to 14 days) after the co-cultivation step on a medium lacking a selection compound. Establishment of a reliable resistance level against the selection compound may need some time to prevent unintended damage by the selection compound.

Transformed cells, i.e. those which comprise the DNA integrated into the DNA of the host cell, can be selected from untransformed cells. As soon as a transformed plant cell has been generated, an intact plant can be obtained using methods known in the art. Callus may be used as target tissue. Preferably, target tissue may be shoots, shoot tips, embryos, embryo-producing tissue, and/or shoot-producing tissues. Plants can then be induced in this tissue in the known fashion. The shoots obtained can be planted and cultured.

Agrobacterium-mediated techniques typically may result in gene delivery into a limited number of cells in the targeted tissue. Therefore, in a preferred embodiment, a selective agent is applied post-transformation to kill all of the cells in the targeted tissues that are not transformed or to identify transformed cells through a selective advantage. The length of culture depends, in part, on the toxicity of the selection agent to untransformed cells. The selectable marker gene and the corresponding selection compound used for said selection or screening can be any of a variety of well-known selection compounds, such as antibiotics, herbicides, or D-amino acids. The length of this culture step is variable (depending on the selection compound and its concentration, the selectable marker gene), extending from one day to 120 days. Insertion of a selectable and/or screenable marker gene is comprised within the scope of the methods of the disclosure. This may be advantageous e.g., for later use as an herbicide-resistance trait.

For example, with the kanamycin resistance gene (neomycin phosphotransferase II, NPTII) as the selective marker, kanamycin at a concentration of from about 3 to 200 mg/L may be included in the medium. Typical concentrations for selection are 5 to 50 mg/L. The tissue is grown upon this medium for a period of 1 to 8 weeks, preferably about 2-4 weeks, until shoots or embryos have developed.

For example, with the phosphinothricin resistance gene as the selective marker, phosphinothricin at a concentration of from about 3 to 200 mg/L may be included in the medium. Typical concentrations for selection are 5 to 50 mg/L. The tissue is grown upon this medium for a period of 1 to 8 weeks, preferably about 2-4 weeks until shoots have developed.

For example, with the dao1 gene as the selective marker, D-serine or D-alanine at a concentration of from about 3 to 100 mg/L may be included in the medium. Typical concentrations for selection are 20 to 40 mg/L. The tissue is grown upon this medium for a period of 1 to 8 weeks, preferably about 2-4 weeks until shoots have developed.

For example, with the hygromycin resistance gene (hygromycin phosphotransferase) as the selective marker, hygromycin at a concentration of from about 3 to 200 mg/L may be included in the medium. Typical concentrations for selection are 5 to 50 mg/L. The tissue is grown upon this medium for a period of 1 to 8 weeks, preferably about 2-4 weeks until shoots have developed.

Transformed plant cells, derived by any of the above transformation techniques, can be cultured to regenerate a whole plant which possesses the transformed genotype and thus the desired phenotype. Such regeneration techniques rely on manipulation of certain growth regulators in a tissue culture growth medium, typically relying on a biocide and/or herbicide marker that has been introduced together with the desired nucleotide sequences. Regeneration can also be obtained from plant callus, explants, somatic embryos, organs, or parts thereof. Plant regeneration from cultured protoplasts is known in the art. Such regeneration techniques are known in the art.

The media as employed during regeneration and/or selection may be optionally further supplemented with one or more plant growth regulator, like e.g., cytokinin compounds (e.g., 6-benzylaminopurine) and/or auxin compounds (e.g., 2,4-D). The term “plant growth regulator” (PGR) as used herein means naturally occurring or synthetic (not naturally occurring) compounds that can regulate plant growth and development. PGRs may act singly or in consort with one another or with other compounds (e.g., sugars, amino acids). The term “auxin” or “auxin compounds” comprises compounds which stimulate cellular elongation and division, differentiation of vascular tissue, fruit development, formation of adventitious roots, production of ethylene, and when present in high concentrations, induce dedifferentiation (callus formation). The most common naturally occurring auxin is indoleacetic acid (IAA), which is transported polarly in roots and stems. Synthetic auxins are used extensively in modern agriculture. Auxin compounds comprise indole-3-butyric acid (IBA), naphthaleneacetic acid (NAA), and 2,4-dichlorophenoxyacetic acid (2,4-D). Compounds that induce shoot formation include, but not limited to, IAA, NAA, IBA, cytokinins, auxins, kinetin, and thidiazuron.

The term “cytokinin” or “cytokinin compound” comprises compounds which stimulate cellular division, expansion of cotyledons, and growth of lateral buds. They delay senescence of detached leaves and, in combination with auxins (e.g. IAA), may influence formation of roots and shoots. Cytokinin compounds comprise, for example, 6-isopentenyladenine (IPA) and 6-benzyladenine/6-benzylaminopurine (BAP).

Descendants can be generated by sexual or non-sexual propagation. Non-sexual propagation can be realized by introduction of somatic embryogenesis by techniques well known in the art. Preferably, descendants are generated by sexual propagation/fertilization. Fertilization can be realized either by selfing (self-pollination) or crossing with other transgenic or non-transgenic plants. Transgenic plants describe herein may function either as maternal or paternal plant. Descendants may comprise one or more copies of the agronomically valuable trait gene. Preferably, descendants are isolated which only comprise one copy of said trait gene.

Kits

Particular embodiments are directed to kits useful for the practice of one or more of the methods described herein. As used herein, a “kit” is any manufacture (e.g. a package or container) comprising at least one sample, e.g. a strain of Agrobacterium, for use in Agrobacterium-mediated transformation of a plant cell, plant tissue, or plant.

In a particular embodiment, a kit comprises at least one aliquot or sample of Agrobacterium strain JTND, or SBHT. In this particular embodiment, the Agrobacterium strain contains a Ti-plasmid capable of integrating DNA into a plant genome. In another particular embodiment, a kit comprises at least one aliquot or sample of Agrobacterium strain JTND, or SBHT, where the Agrobacterium strain comprises a helper plasmid, wherein the helper plasmid comprises a Ti plasmid comprising a vir region of the Agrobacterium, and wherein the helper plasmid lacks a T-DNA region, and at least one separate aliquot or sample comprising a binary plasmid, wherein the binary plasmid comprises a T-DNA region. In this embodiment, the T-DNA region of the binary plasmid preferably comprises right and left border sequences and minimal internal sequences. More preferably, the T-DNA region of the binary plasmid comprises no other T-DNA other than the right and left border sequences.

In another embodiment, the kit described herein further comprises additional elements, including but not limited to appropriate growth media, antibiotics useful for eliminating Agrobacterium following transformation, and a selection reagent capable of selecting for transgenic plant cells following transformation. In addition, the kits of the present invention may preferably contain instructions which describe a suitable detection assay. Such kits can be conveniently used, e.g., in laboratory settings, to transform plants with a gene of interest, regulatory gene of interest, selectable marker gene, reporter gene, or combinations thereof.

The kits described herein reduce the costs and time associated transforming a variety of plants, most particularly Glycine max (soybean). The kits may be used by research and commercial laboratories and agro-biotechnology companies to facilitate plant variety generation through Agrobacterium-mediated transformation.

Certain aspects and embodiments of the invention will now be illustrated by way of example with reference to the figures.

### EXAMPLES

### Example 1

### Materials and Methods for Producing Useful Agrobacterium Compositions and Plants Produced Using the Compositions

Isolation of Agrobacterium from Soil Samples

Soil samples were collected from various locations across Ohio and the U.S. Soil samples (1 g) were suspended in 5 mL sterile water and the suspension was vortexed at medium speed in a Thermo Fisher Scientific Vortex Genie 2 (Model G-560; Thermo Fisher Scientific; Waltham, Mass.) table top unit. Suspensions were serially diluted to 10⁻¹, 10⁻², 10⁻³ and 10⁻⁴, and 300 μL was spread aseptically on 1A semi-selective medium (Table 1) in 100×15 mm Petri plates. Plates were incubated at 28° C. for a minimum of 2 d, or until colonies of at least 2 mm in diameter were present.

TABLE 1
1A Recipe - Semi-selective medium for Agrobacterium spp.;
w/v, per 1000 mL.

| | | |
|---|---|---|
| L (—) arabitol | 3.04 | g |
| NH₄NO₃ | 0.16 | g |
| KH₂PO₄ | 0.54 | g |
| K₂HPO₄ | 1.04 | g |
| Sodium taurocholate | 0.29 | g |
| MgSO₄•7H₂O | 0.25 | g |
| Agar | 15.0 | g |
| Crystal violet, 0.1% (w/v) | 2.0 | mL |
| Cycloheximide, 2% | 1.0 | mL |
| Na₂SeO₃, 1% | 6.6 | mL |
| K₂TeO₃ | 80 | mg |

Putative colonies which appeared shiny and black were picked with sterile filter pipette tips and the tips were swirled in both 15 μL liquid Yeast Extract Peptone (YEP) medium (10 g/L peptone, 10 g/L yeast extract, 5 g/L NaCl, pH 7.0) and 15 μL H₂O, each contained in separate 96 multiwell plates. Multiwell plates containing YEP were incubated for 6-8 h at room temperature and gently shaken at 150 RPM. A mixture of 0.5% (w/v) sodium azide and 2% (v/v) Triton X 100 was then placed into each well in a fume hood, to reach a final percentage of 0.5% sodium azide and 1% Triton X 100. The sodium azide was included as a lysis agent and Triton X 100 as a detergent to increase lysis activity.

Lysed bacteria were heated in the 96 well PCR reaction vessel in a BioRad Laboratories PCR machine (Model # T100; BioRad Laboratories, Hercules, Calif.) at 95° C. for 10 min and then cooled to 10° C. for 5 min. Samples were centrifuged at 6,000×g for 30 min in a table top Sorvall Legend RT Refrigerated Centrifuge (Model #75004377; Thermo Fisher Scientific, Waltham, Mass.). A 0.5 μL sample of supernatant was removed from each well, and touchdown PCR was run with the virG primer set (Table 2) and GoTaq Green Master Mix (Model # M3001, Promega Corporation, Fitchburg, Wis.) using the following conditions: denaturation for 5 min at 95° C., then using a cycle profile of 94° C. for 30 s, then cycle 5 times for each degree, 62° C.-57° C. for 1 min, then 72° C. 1 min, 72° C. for 10 min and then hold at 15° C. DNA amplicons were analyzed using a 1.5% agarose gel stained with ethidium bromide and visualized under UV illumination.

TABLE 2
Oligonucleotide primers used in this study

| | | Length | |
|---|---|---|---|
| Name | Primer sequence (5′-3′) | (bp) | Target |
| VirG | ATcTYAATTTRggKcgYgAAgA | 539 | Virulent |
| | (SEQ ID NO: 1) | | strain of A. |
| | cAcRTcMgcgTcRAAgAAATA | | tumefaciens |
| | (SEQ ID NO: 2) | | |
| AGRH | gccRcgccAgATcAAcAgYWc | 739 | Mixed Glade |
| | (SEQ ID NO: 3) | | Agrobacterium/ |
| | ATcSAgRTcRTgNcccATcg | | Rhizobium |
| | (SEQ ID NO: 4) | | |
| ResTin | AcccAcggTTcTccTTcgcATAg | 473 | Biovar 1 |
| | (SEQ ID NO: 5) | | |
| | ccTgAccgTTgAAcAggATgcTc | | |
| | (SEQ ID NO: 6) | | |
| M13F/ | cccAgggTTTTcccAgTcAcgAc | ~500 | pCAMBIA insert |
| sGFPR | (SEQ ID NO: 7) | | |
| | ccgTAggTggcATcgc | | |
| | (SEQ ID NO: 8) | | |

Colonies which yielded amplicons following PCR were then referenced back to the YEP 96 well plate, and a sample was removed for a second round of PCR for validation. In addition, the suspension was re-streaked on 1A medium (FIG. 6). Positive strains were re-streaked on 1A medium a minimum of three successive times and tested again for virG. Colonies positive after this were considered bona fide Agrobacterium.

Isolation of Agrobacterium from Plant Galls

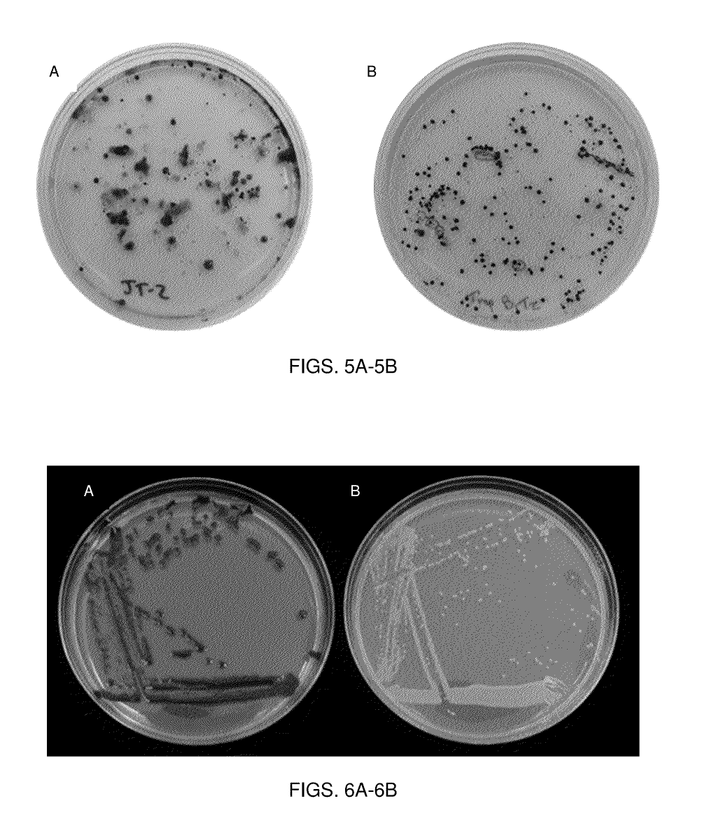

Galls were obtained from chrysanthemum (Chrysanthemum indicum), euonymus (Euonymus obovatus), and rose (Rosa sp.). Samples of tumor tissues (0.5 g) were ground with a micropestle in microfuge tube containing 1 mL sterile water and incubated overnight at 26° C. Gall extracts were serially diluted to 10⁻¹, 10⁻², 10⁻³ and 10⁻⁴, and 300 μL was spread aseptically on 1A medium. Plated extracts were incubated at 28° C. for 48 h. In the same manner of selection of colonies from soil, Agrobacterium colonies, which had grown to 2 mm in diameter with a dense, shiny black morphology on 1A medium (FIG. 5) were selected for isolation and PCR.

Introduction of pCAMBIA1300-Gmubi3

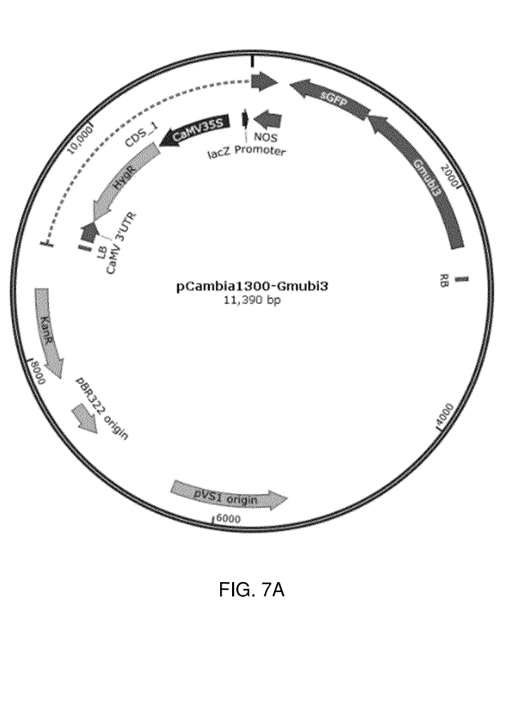

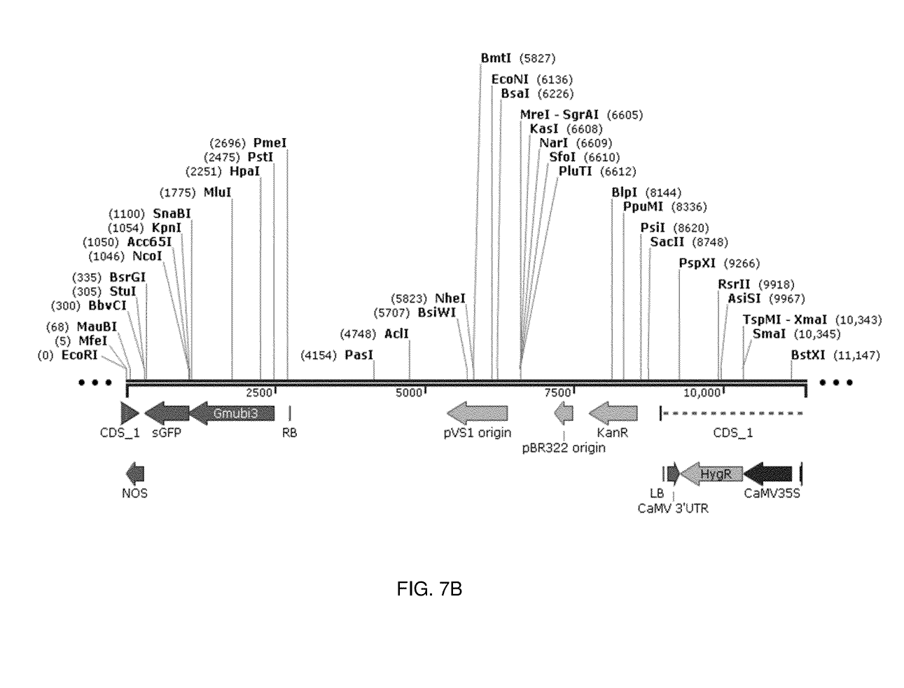

The binary plasmid pCAMBIA1300 (Cambia, GenBank accession number AF234296) modified to contain GFP driven by the Gmubi3 promoter (pCAMBIA-Gmubi3, FIG. 7a), was introduced into all Agrobacterium strains via freeze/thaw transformation. All transformed isolates were selected on YEP medium containing 50 mg/L kanamycin plate, except for strain KFOH, which was selected on medium containing 50 mg/L hygromycin. Colony PCR was performed with primer set M13F and sGFPR (Table 2) to confirm successful plasmid uptake.

Single colonies were picked and re-streaked a minimum of three times on a selective medium, after which colony PCR was run again using the virG and the M13/sGFP primer sets (Table 2).

Preparation of Agrobacterium for Plant Transformation

Agrobacterium strains were streaked onto YEP medium containing antibiotics 4 d prior to transformation. After 2 d, single colonies were picked and grown overnight in 5 mL liquid YEP containing antibiotics at 28° C. Approximately 1 mL of the overnight culture was used to inoculate 30 mL liquid YEP for an additional overnight culture.

The 30 mL liquid cultures were transferred to 50 mL plastic conical tubes and centrifuged at 3,000 g for 10 min. The supernatant was discarded and the bacterial pellet was re-suspended in liquid MS medium, containing Murashige and Skoog salts, Gamborg's B5 vitamins and 3% sucrose (pH 5.7). After the OD₆₀₀ was adjusted to 0.5-0.75, AS was added to the bacterial suspensions to a final concentration of 100 μM. The bacterial suspensions were then allowed to sit at room temperature for about 30 min.

Plant Materials

Agrobacterium strains were evaluated for transformation efficiency using seedling materials of sunflower (cv. ‘RHA280’) and soybean (cv. ‘Thorne’). Seeds were surface sterilized with a 4% (v/v) bleach solution for 10 min with shaking at 150 rpm, and rinsed 5× with sterile water, until the odor of bleach was no longer present. Seeds were germinated in between water-saturated sterile paper towels in Magenta culture vessels (Model # GA-7, Magenta Corp., Chicago, Ill.) for 5 d at 25° C. before use for transformation.

Soybean embryogenic suspension cultures were initiated from Glycine max cv. ‘Thorne’, on D40 medium and maintained in 30 mL FN medium in 50 mL baffled flasks, shaken at 150 RPM at 25° C. Suspension culture tissue was subcultured every 2-3 wk, using 5-10 pieces of rapidly proliferating embryogenic tissue, taken about 1 wk prior to transformation

Plant Transformation

Seedlings were placed into a sterile Petri dish containing 5 mL of bacterial suspension in MS medium with AS and dissected directly in the solution. For preparation of cotyledons, both ends of cotyledons were excised and discarded, and the remaining cotyledon segments were then cut into 2-3 mm cross sections. For preparation of stem segments, roots were removed and hypocotyls were cut into 2-3 mm sections. After dissection, explants were blotted on dry filter paper, and immediately placed onto solid co-culture medium containing MS salts, B5 vitamins, and 3% sucrose. For soybean tissues, the solid co-culture medium was modified to contain 100 μM AS and 500 mg/L DTT. Tissues were co-cultured for 2 d at 25° C., and then transferred to medium containing MS salts, B5 vitamins, 3% sucrose and 400 mg/L Timentin for 3 d. GFP expression was measured 5 d after initial inoculation or 3 d after transfer of the tissue to the medium containing Timentin.

For soybean suspension cultures, 10 clumps of embryogenic tissue were placed in 13×100 mm borosilicate thin walled glass test tubes containing 5 mL bacterial suspension. Tissues in the tubes were sonicated for 10 s, while keeping tissue at least 3 mm below the water surface in the water bath. The bacterial solution was then discarded and the embryogenic tissue was blotted on sterile filter paper to remove excess bacteria.

Embryogenic tissues were co-cultured in liquid FN medium containing 100 μM AS and 500 mg/L cysteine for 2 d, and then transferred to fresh FN medium containing 400 mg/L Timentin and 500 mg/L-cysteine for 3 d. GFP expression was quantified by counting the total number of GFP foci per tissue clump using a MZFLIII stereomicroscope (Leica, Heerbrugg, Switzerland) and averaged per treatment.

Calculations and Data Analysis

Each experiment was performed as three separate biological replicates for each treatment. Mean GFP expressing foci counts were calculated per cut side of hypocotyl or cotyledon explants and embryogenic tissue clumps 2-3 mm in size. Data was analyzed in R (Version #3.0.3, R Core Team, Vienna, Austria) and Minitab (Version #16.0, Minitab Inc., State College, Pa.) using ANOVA procedure and PROC GLM. Means separation was carried out using Fisher's test.

### Example 2

### Manipulation and Laboratory Alterations of Agrobacterium and Plants

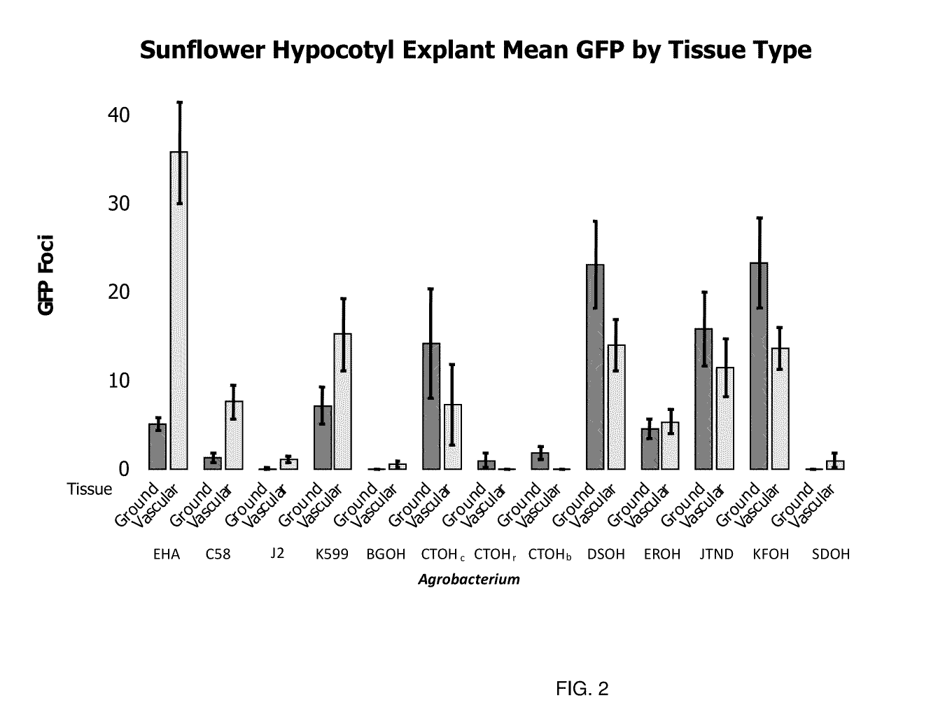

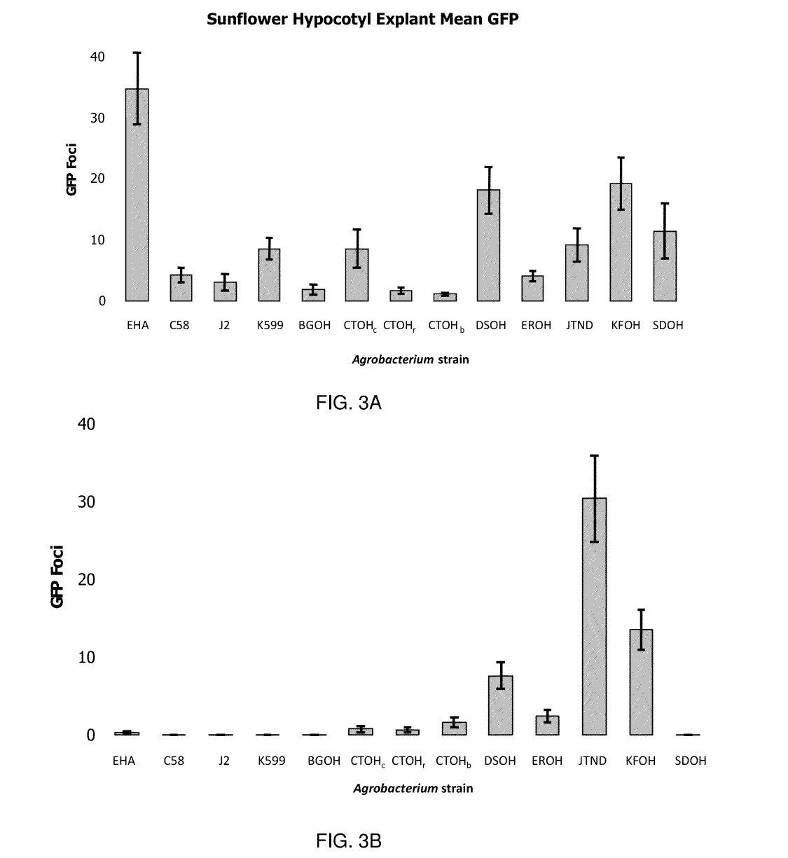

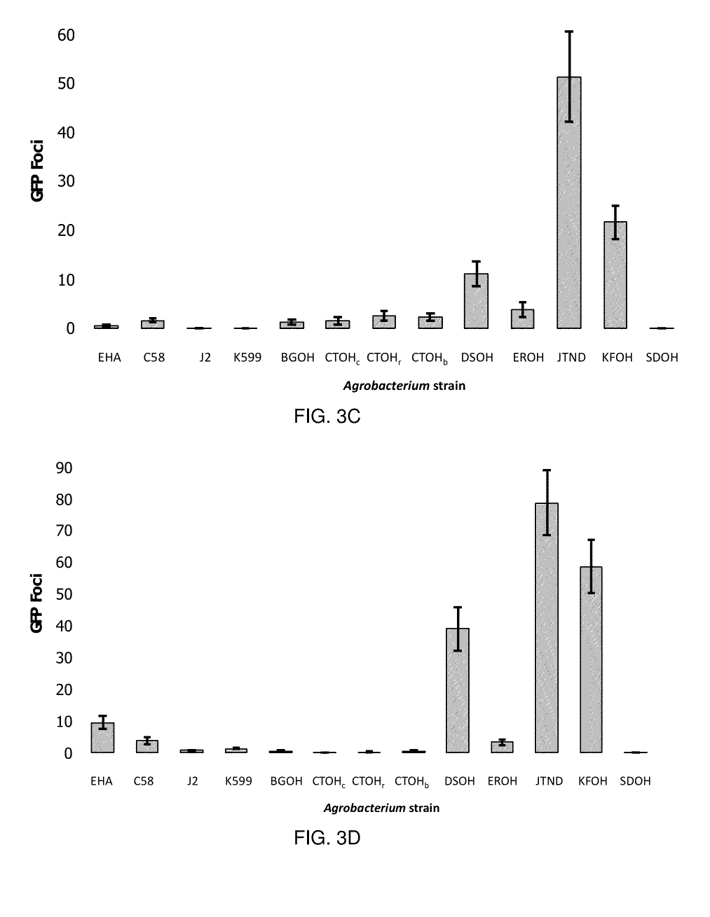

pCAMBIA Gmubi3 (FIG. 7a) was introduced into thirteen bacterial strains Transformation was evaluated and quantified in plants using sunflower and soybean seedling tissue and proliferative embryogenic soybean tissue with GFP expression. The efficiency of transformation was measured as number of GFP expressing foci per explant or embryogenic clump of tissue. Tissue types transformed were significantly affected by the bacterial strain used (FIGS. 2, 3B, 3C, 3D). The wild-type novel strains had a larger number of average foci than with known strains.

Five Agrobacterium strains were isolated from soil collected from soybean fields in the US, one strain was collected from soil from a creek bed and three strains were collected from the galls of Chrysanthemum indicum, Euonymus obovatus, and Rosa sp. (Table 3, 4). Since crown gall has never been reported in soybean fields, isolation of Agrobacterium from soybean galls could not be undertaken. The inventors successfully produced strains with enhanced transformation attributes for soybean. This “host-enhanced” Agrobacterium-mediated transformation tool was useful for expression of foreign nucleic acid, including genes.

TABLE 3
The first nine listed strains were isolated in the course of the work
described herein; soybean field isolates were collected for the
purpose of comparing to the current top performing strains available,
and gall isolates collected for a comparison to other WT isolates.

| Strain | Sample location | Substrate isolated from |
|---|---|---|
| SDOH | Sandusky, OH | Soybean field |
| DSOH | Central OH | Soybean field |
| EROH | Erie, OH | Soybean field |
| JTND | Hillsboro, ND | Soybean field |
| CTOHb | Ottawa, OH | Soybean field |
| KFOH | Wooster, OH | Creek bed soil |
| BGOH | Hiram, OH | Euonymus gall |
| CTOHr | Wooster, OH | Rose gall |
| CTOHc | Wooster, OH | Chrysanthemum gall |
| EHA105 | Lab strain | Cherry gall |
| C58 | Lab strain | Cherry gall |
| J2 | Lab strain | Cherry gall |
| K599 | Lab strain | Cucumber |

Evaluation of Transformation

Transformation assays targeting seedling-derived explants of soybean and sunflower were carried out in a manner approximating the cotyledonary node transformation system. The number of cells expressing GFP within each explant is reported. For inoculation of embryogenic cultures, sonication was used to micro-wound the embryos, allowing for more access points for the bacterium to infect host cells. In addition, wounded tissues produce phenolics which trigger the T-DNA transfer process. Embryo sonication and explant excising act to activate this phenolic compound production and has been shown to increase transformation in many tissues. Because of the ability to inhibit oxidation, reducing agents including DTT and L-cysteine have been used to minimize the appearance of necrosis, leading to increased transgene expression.

Sunflower Transformation

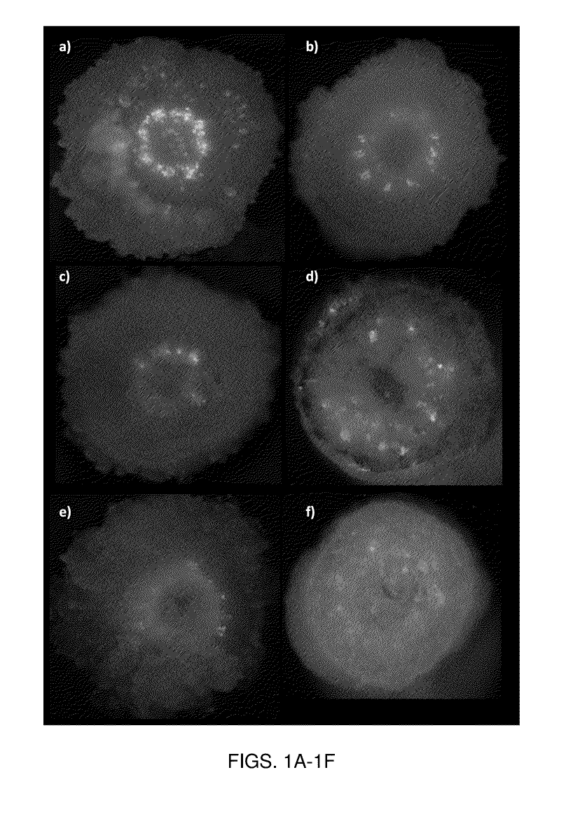

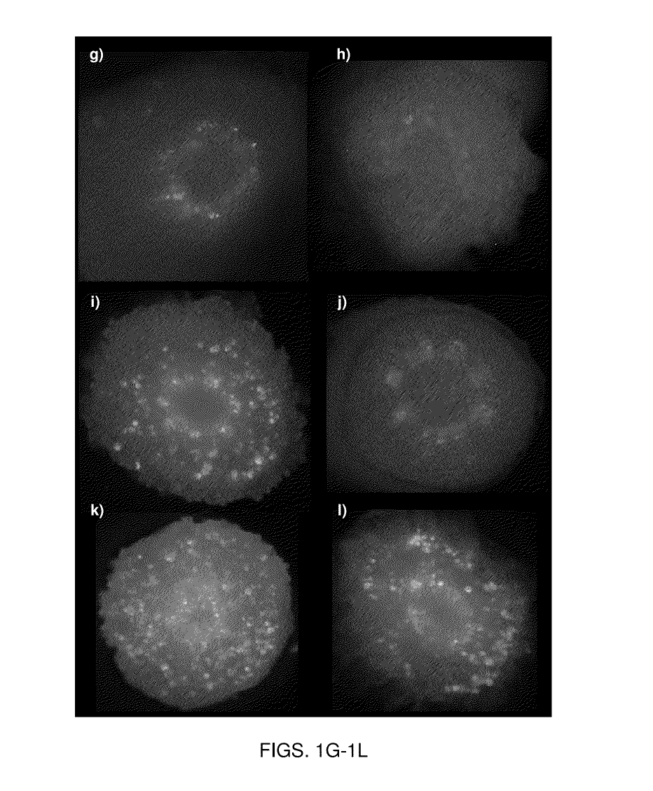

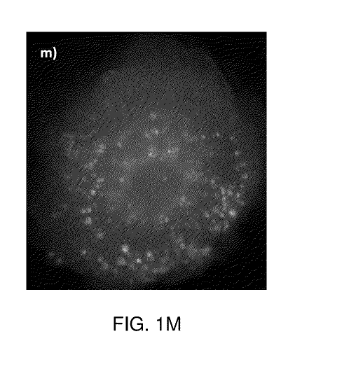

In sunflower seedling explants using the pCAMBIA1300 Gmubi3 binary plasmid, some tissues were observed to be more responsive to transformation using previously known laboratory strains (FIG. 1). EHA105 had the highest overall transformation efficiency (FIG. 3A). Use of EHA105 gave higher transformation rates in vascular tissues compared to the soil-derived strains which showed no such preference for tissue specific transformation and GFP foci were evenly distributed throughout the tissues (FIG. 2). Further analysis of tissue-specific transformation in sunflower hypocotyls revealed that the C58 strain also showed a preference for vascular tissue targeting.

Soybean Transformation

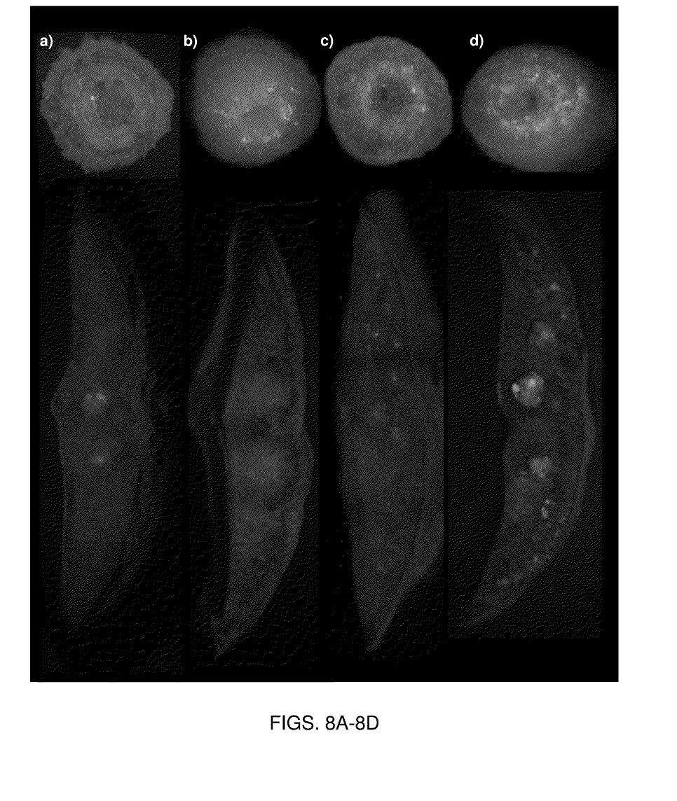

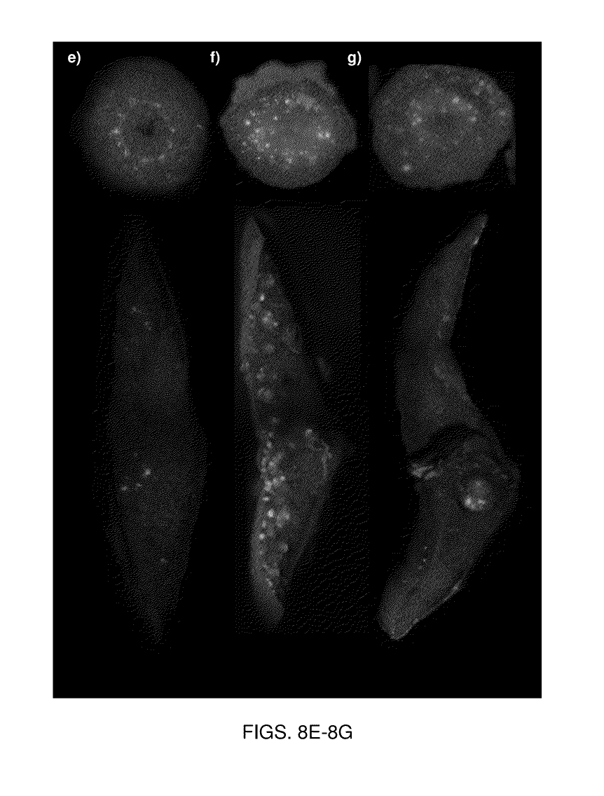

In soybean hypocotyl explants, cotyledon explants and embryogenic tissues, use of strain EHA105 resulted in a very low efficiency of transformation (FIG. 8). Although this strain showed high efficiency transformation of sunflower hypocotyls, this same strain worked very inefficiently for transformation of all soybean tissues, based on GFP foci counts.

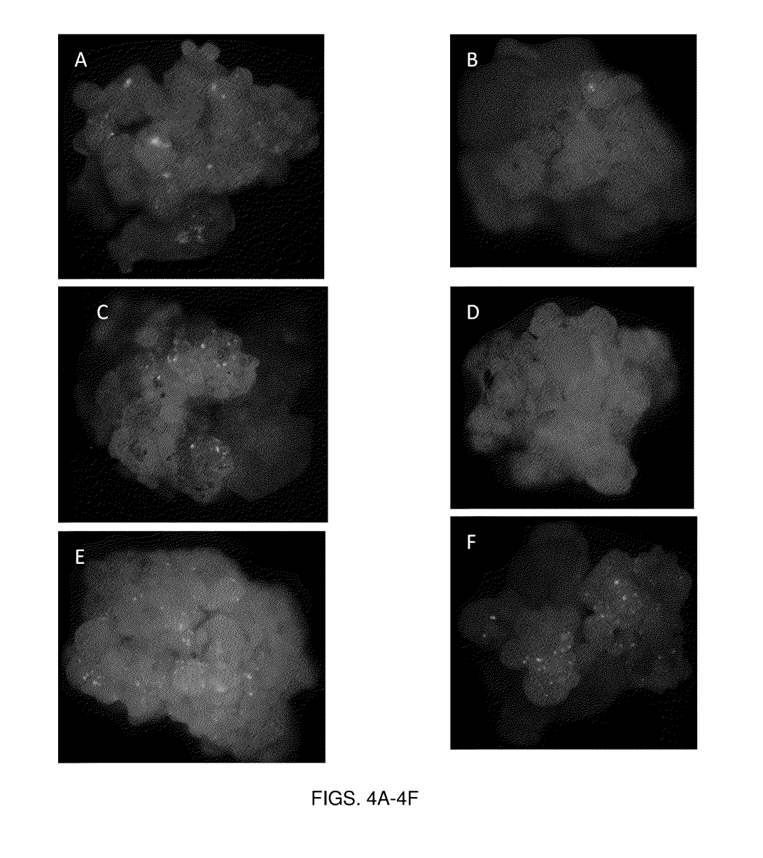

Of the strains that were obtained, three soil-derived strains showed improved transformation rates in all soybean tissues tested. Interestingly, strains produced a consistent transformation profile across all soybean tissues; the more efficient strains gave high transformation rates with soybean hypocotyls (FIG. 3B), cotyledons (FIG. 3C) and embryogenic cultures (FIGS. 3D, 4). The least effective strains showed low transformation rates in all soybean tissues. In addition, tissue-specific transformation of vascular versus ground tissue in hypocotyls and cotyledons of soybean was not observed. Agrobacterium strains JTND and KFOH gave the highest transformation efficiency in all soybean tissues tested.

Deposit Information

A deposit of the Agrobacterium tumefaciens strain JTND of the invention is maintained by Dr. John J. Finer, Department of Horticulture and Crop Science, The Ohio State University, 1680 Madison Ave., Wooster, Ohio 44691. Access to this deposit will be available upon written request to persons determined by the Commissioner of Patents and Trademarks to be entitled thereto under 37 CFR §1.14 and 35 USC §122.

Applicant will deposit the Agrobacterium tumefaciens strain JTND of the invention with the American Type Culture Collection (ATCC), Manassas, Va., in compliance with the Budapest Treaty and in compliance with 37 C.F.R. §§1.801-1.809. The ATCC Accession No. will be provided upon receipt thereof. Following deposit with the ATCC, access to this deposit will be available during the pendency of this application to persons determined by the Commissioner of Patents and Trademarks to be entitled thereto under 37 CFR §1.14 and 35 USC §122.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

What is claimed is:

1. An isolated Agrobacterium strain JTND.

2. The isolated Agrobacterium strain JTND of claim 1, wherein the isolated strain is substantially biologically pure.

3. A disarmed Agrobacterium strain, wherein the disarmed Agrobacterium strain is a disarmed JTND strain.

4. A disarmed Agrobacterium strain SBHT.

5. The disarmed Agrobacterium SBHT strain of claim 4, wherein the strain is substantially biologically pure.

6. A method of producing a transgenic plant cell, transgenic plant tissue, or transgenic plant, comprising the steps:
a) providing an Agrobacterium strain selected from the group consisting of: JTND; and SBHT, wherein the Agrobacterium strain comprises a transgenic T-DNA region; and
b) contacting the Agrobacterium with a plant cell, plant tissue, or plant under conditions that permit the Agrobacterium to transform the plant cell, plant tissue, or plant, thereby producing a transgenic plant cell, transgenic plant tissue, or transgenic plant.

7. The method of claim 6, wherein the transgenic T-DNA comprises at least one plant-expressible gene of interest.

8. The method of claim 6, wherein the transgenic T-DNA comprises at least one regulatory gene of interest.

9. The method of claim 6, wherein the transgenic T-DNA comprises at least one genome editing nuclease.

10. The method of claim 9, wherein the at least one genome editing nuclease is selected from the group consisting of: Zinc-finger nucleases (ZFNs); transcription activator-like effector nucleases (TALENs); and clustered regularly interspaced short palindromic repeat (CRISPR)/Cas-based RNA-guided DNA endonucleases.

11. The method of claim 7, wherein the at least one plant-expressible gene of interest comprises one or more genes associated with at least one agronomically valuable trait selected from the group consisting of: increased yield; drought tolerance; cold tolerance; heat tolerance; salt tolerance; increased nutrient content; reduced development time; increased vigor; herbicide resistance; and pest resistance.

12. The method of claim 6, further comprising isolating or selecting a plant cell, plant tissue, or plant comprising the transgenic T-DNA.

13. The method of claim 12, further comprising regenerating a plant from the transgenic plant cell or plant tissue comprising the transgenic T-DNA.

14. The method of claim 13, further comprising collecting seed from the regenerated transgenic plant.

15. The method of claim 14, wherein the collected seed comprises the transgenic T-DNA region.

16. The method of claim 13, further comprising:
a) selfing the transgenic plant or crossing the transgenic plant with a second plant; and
b) selecting resulting progeny comprising the transgenic T-DNA region.

17. The method of claim 16, wherein resulting progeny are further selected for having an agronomically valuable trait conferred by the transgenic T-DNA region.

18. The method of claim 6, wherein the transgenic T-DNA is stably integrated into the genome of the plant cell, plant tissue, or plant.

19. The method of claim 6, wherein the transgenic T-DNA region comprises at least one plant-expressible selectable marker gene.

20. The method of claim 6, wherein the step of contacting the Agrobacterium with the plant cell, plant tissue, or plant is accomplished by at least one method selected from the group consisting of: incubating the plant cell, plant tissue, or plant with Agrobacterium; co-cultivation of the at least one plant host cell and the Agrobacterium; floral dip method; vacuum infiltration method; cotyledonary-node method; and sonication-assisted Agrobacterium-mediated transformation, or a combination thereof.

21. The method of claim 6, wherein the plant tissue is selected from the group consisting of: immature plant embryo; mature plant embryo; seed; seedling; root; cotyledon; stem; node; internode; bud; leaf; shoot apical meristem; and cultured plant material.

22. The method of claim 6, wherein the plant cell is a cell from a plant part selected from the group consisting of: pollen; ovule; immature plant embryo; mature plant embryo; seed; seedling; root; cotyledon; stem; node; internode; bud; leaf; shoot apical meristem; and cultured plant material.

23. The method of claim 6, wherein the plant cell or plant tissue is from a plant selected from the group consisting of: monocotyledonous plants; dicotyledonous plants; and gymnosperm plants.

24. The method of claim 6, wherein the plant is selected from the group consisting of:
monocotyledonous plants; dicotyledonous plants; and gymnosperm plants.

25. The method of claim 6, wherein the plant cell or plant tissue is from a plant of a genus selected from the group consisting of: Glycine; Medicago; Pisum; Beta, Helianthus; Arabidopsis; Dioscorea; Ipomea; Manihot; Plantago; Zea; Oryza; Sorghum; Triticum; Hordeum; Saccharum; Brassica; Solanum; Nicotiana; Gossypium; Vitis; Populus; Picea; and Pinus.

26. The method of claim 6, wherein the plant is of a genus selected from the group consisting of: Glycine; Medicago; Pisum; Beta, Helianthus; Arabidopsis; Dioscorea; Ipomea; Manihot; Plantago; Zea; Oryza; Sorghum; Triticum; Hordeum; Saccharum; Brassica; Solanum; Nicotiana; Gossypium; Vitis; Populus; Picea; and Pinus.

27. The method of claim 6, wherein the plant cell or plant tissue is from a plant of the genus Glycine.

28. The method of claim 6, wherein the plant cell or plant tissue is from a soybean plant.

29. The method of claim 6, wherein the plant is of the genus Glycine.

30. The method of claim 6, wherein the plant is a soybean plant.

31. A transgenic plant comprising a plurality of transgenic plant cells of claim 6.

32. A seed or plant part of the transgenic plant of claim 31.

33. A kit for transforming a plant cell, plant tissue, or plant comprising: at least one sample comprising an Agrobacterium strain selected from the group of Agrobacterium strains consisting of: JTND; and SBHT, wherein the Agrobacterium strain comprises a Ti-plasmid.

34. A kit for transforming a plant cell, plant tissue, or plant comprising:
a) at least one sample comprising an Agrobacterium strain selected from the group of Agrobacterium strains consisting of: JTND; and SBHT, wherein the Agrobacterium strain comprises a helper plasmid, wherein the helper plasmid comprises a Ti plasmid comprising a vir region of the Agrobacterium, and wherein the helper plasmid lacks a T-DNA region; and
b) at least one sample comprising a binary plasmid, wherein the binary plasmid comprises a T-DNA region.

35. The kit of claim 34, further comprising at least one sample of growth media.

36. The kit of claim 34, further comprising at least one sample of at least one antibiotic, wherein the at least one antibiotic is capable of eliminating the Agrobacterium strain following transformation of a plant cell.

37. The kit of claim 34, further comprising at least one sample of at least one reagent capable of selecting for transgenic plant cells following transformation of a plant cell.

38. The kit of claim 34, further comprising instructions for use of the kit.

39. A method to make a host-enhanced transformation tool, comprising:
a) obtaining a soil sample from a field where a host plant has grown or is growing;
b) isolating an Agrobacterium strain from the soil sample; and
c) disarming the Agrobacterium strain so as to make a host-enhanced transformation tool.

40. The method of claim 39, which further comprises introducing a foreign nucleic acid into the transformation tool.

41. A host-enhanced transformation tool made according to a method of claim 39.

42. A method to transfer foreign nucleic acid into a host plant, comprising introducing a transformation tool of claim 39 to a host plant, and inducing the transformation tool to transfer the foreign nucleic acid to the host plant.


SEQUENCE LISTING (8 sequences)

SEQ ID NO: 1 - DNA, 22 bp, Artificial Sequence
  Description of Artificial Sequence Synthetic primer
atctyaatttrggkcgygaaga

SEQ ID NO: 2 - DNA, 21 bp, Artificial Sequence
  Description of Artificial Sequence Synthetic primer
cacrtcmgcgtcraagaaata

SEQ ID NO: 3 - DNA, 21 bp, Artificial Sequence
  Description of Artificial Sequence Synthetic primer
gccrcgccagatcaacagywc

SEQ ID NO: 4 - DNA, 20 bp, Artificial Sequence
  Description of Artificial Sequence Synthetic primer
atcsagrtcrtgncccatcg

SEQ ID NO: 5 - DNA, 23 bp, Artificial Sequence
  Description of Artificial Sequence Synthetic primer
acccacggttctccttcgcatag

SEQ ID NO: 6 - DNA, 23 bp, Artificial Sequence
  Description of Artificial Sequence Synthetic primer
cctgaccgttgaacaggatgctc

SEQ ID NO: 7 - DNA, 23 bp, Artificial Sequence
  Description of Artificial Sequence Synthetic primer
cccagggttttcccagtcacgac

SEQ ID NO: 8 - DNA, 16 bp, Artificial Sequence
  Description of Artificial Sequence Synthetic primer
ccgtaggtggcatcgc